(19) United States
(12) Patent Application Publication
(10) Pub. No.: US 2023/0070047 A1
(43) Pub. Date: Mar. 9, 2023

(54) METHOD TO ENHANCE GENE EDITING

(71) Applicant: Regents of the University of Minnesota, Minneapolis, MN (US)

(72) Inventor: Shauna Yuan, Edina, MN (US)

(21) Appl. No.: 17/811,048

(22) Filed: Jul. 6, 2022

Related U.S. Application Data
(60) Provisional application No. 63/218,746, filed on Jul. 6, 2021.

(51) Int. Cl. C12N 15/90; C12N 9/22; C12N 15/10; C12N 5/074; C12M 1/42

(52) U.S. Cl. CPC C12N 15/907; C12N 9/22; C12N 15/102; C12N 5/0696; C12M 35/02; C12N 2310/20

(57) ABSTRACT

A method to enhance homology directed recombination (HDR) efficiency, and a kit therefor, are provided.

### CROSS-REFERENCE TO RELATED APPLICATIONS

This application claims the benefit of the filing date of U.S. application No. 63/218,746, filed on Jul. 6, 2021, the disclosure of which is incorporated by reference herein.

### STATEMENT OF GOVERNMENT RIGHTS

This invention was made with government support under AG070415 awarded by the National Institutes of Health. The government has certain rights in the invention.

### BACKGROUND

Although genome editing by CRISPR has significantly improved the homology derived recombination (HDR) efficiency for genetic engineering, there are still some bottlenecks. Some sites are difficult to target, often due to their location on the chromosome. There can also be cell-line variability, for some cell lines may not be able to withstand the stress from electroporation or a double-stranded break caused by Cas9 (Budde et al., 2017; Conti & Di Micco, 2018). Pluripotent stem cells (PSC), including induced pluripotent stem cells (iPSC), allow for the study human development, diseases and drug screening in vitro, and are now widely used in laboratories. CRISPR's ability to create genetically engineered iPSC lines provides a potent tool to utilize the PSCs with isogenic controls. However, there is still a pressing need for more efficient CRISPR protocols, e.g., those designed to work with PSCs.

The CRISPR editing toolkit has been improving. The discovery of asymmetric ssODN (single strand oligonucleotide) has improved targeting efficiency and the design of the donor template (Richardson et al., 2016). Findings about insertions and deletions (INDEL) have shown how they can be reduced by altering the protospacer adjacent motif (PAM) site to prevent re-editing, which improves the HDR efficiency (Paquet et al., 2016; Kwart et al., 2017). Excessive cell death after nucleoporation can result in poor overall cell recovery, thus resulting in a low number of cells for screening and a low yield of homologous recombinant clones (Budde et al., 2017; Li et al., 2018). Cas9 activates p53 and causes cell death via activation of apoptosis (Enache et al., 2020). Inhibition of apoptosis via inhibition of p53 has been tested in pluripotent stem cells (Li et al., 2018) for selection of CRISPR edited clones.

Higher editing efficiency has recently been reported, by using modified ssODN, sgRNA, heat shock and drug selection. Li et al. transfected plasmids into iPSC to express Cas9 and sgRNA and for p53 inhibition. They found that electroporation was the main factor causing cell death. Their use of p53 inhibition improved the HDR rate up to 50% and so improved cell survival. Drug selection further improved the HDR rate (Li et al., 2018). Skarnes et al. used pre-complexed sgRNA, modified ssODN, heat shock and a HDR enhancer and reported a HDR rate near 70% for one single locus (Skarnes et al., 2019). Kagita et al. found that different electroporation setups had different cell death rates. They also found that precomplexed sgRNA had better editing efficiency than electroporating the CRISPR enzyme and sgRNA plasmids. By optimizing the electroporation setup and the use of precomplexed sgRNA, they saw near 70% HDR efficiency at multiple loci (Kagita et al., 2021).

However, low HDR efficiency and low cell survival continue to be bottlenecks for the iPSC gene editing.

### SUMMARY

The disclosure provides a method which improves gene editing, e.g., CRISPR editing, zinc finger nuclease (ZFN) editing or transcription activator like effector nuclease (TALEN) editing, efficiency by using factors that enhance cell survival and homology directed recombination (HDR). For example, in one embodiment, the HDR rate is enhanced from about 5% up to about 65%. In one embodiment, the HDR rate is enhanced from about 5-foold to about 30-fold.

In one embodiment, the disclosure provides a method to enhance homology directed recombination (HDR) efficiency, comprising: combining mammalian cells, an electroporation enhancer, a recombinase, and a single stranded oligonucleotide donor template comprising one or more homology arms for the selected target, and optionally an inhibitor of p53, thereby providing a mixture; subjecting the mixture to electroporation conditions; and incubating the electroporated cells with a HDR enhancer and a composition comprising one or more cell survival factors for a period of time so as to provide for enhanced homologous recombination at the target in the electroporated cells relative to a corresponding mixture that lacks one or more of the electroporation enhancer, the HDR enhancer or one or more of the cell survival factors.

In one embodiment, a method to enhance homology directed recombination (HDR) efficiency is provided that includes combining mammalian cells, an electroporation enhancer, a ribonucleoprotein complex comprising gRNA or sgRNA for a selected target in the genome of the cells and Cas polypeptide or gRNA or sgRNA for a selected target in the genome of the cells and Cas polypeptide or a vector encoding Cas polypeptide, the sgRNA or the gRNA, a vector for p53 inhibition, and a single stranded oligonucleotide donor template comprising one or more homology arms for the selected target, thereby providing a mixture; subjecting the mixture to electroporation conditions; incubating the electroporated cells with a HDR enhancer and a cell survival factor for a period of time so as to provide for enhanced homologous recombination at the target in the electroporated cells relative to a corresponding mixture that lacks one or more of the electroporation enhancer, the vector for p53 inhibition, the HDR enhancer or the cell survival factor(s). In one embodiment, the method further comprises incubating the electroporated cells of in a medium that includes a cell survival factor but not a HDR enhancer. In one embodiment, the method includes providing a mixture comprising mammalian cells, a ribonucleoprotein complex comprising gRNA or sgRNA and Cas polypeptide, a vector for p53 inhibition and a single stranded oligonucleotide donor, and an electroporation enhancer; subjecting the mixture to electroporation conditions; and incubating the electroporated cells with a HDR enhancer and a cell survival factor for a period of time so as to provide for homologous recombination at the target in the electroporated cells. In one embodiment, the cells are stem cells. In one embodiment, the cells are pluripotent stem cells. In one embodiment, the cells are induced pluripotent stem cells. In one embodiment, the cells are human cells. In one embodiment, the electroporation enhancer comprises nucleic acid or NATE™. In one embodiment, the electroporation enhancer comprises TTAGCTCTGTTTACGTCCCAGCGGGCATGAGAGTAACAAGAGGGTGTGG TAATATTACGGTACCGAGCACTATCGATACAATATGTGTCATACGGACA CG (SEQ ID NO:1), the complement thereof, or a nucleic acid sequence having at least 80%, 82%, 84%, 85%, 86%, 88%, 90%, 92%, 94%, 95%, 96%, 97%, 98% or 99% nucleotide sequence identity thereto. In one embodiment, the homology arms of the single stranded oligonucleotide are about 30 to about 50 nucleotides in length. In one embodiment, the cell survival factor comprises CloneRTM, ROCK inhibitor or Revital Cell Supplement, Y-27632 or fasudil. In one embodiment, the cell survival factors comprise a cocktail of compounds, e.g., having chroman 1, emricasan, polyamines, and trans-ISRIB. In one embodiment, the method further includes cloning or otherwise isolating one or more of the electroporated cells, e.g., in the absence of selection. In one embodiment, the HDR enhancer comprises SCR7, RS-1, L755507, RS1, nocodazole, brefeldin A, 3′-azido-3′deoxythymidine, (Z)-4-hydroxytamoxifen or Alt-RTM HDR enhancer, NU7026, Trichostatin A, MLN4924, NSC 19630, NSC 15520, AICAR, Resveratrol, STL127685, or B02. In one embodiment, the homologous recombination efficiency is enhanced by at least 5 times that of the corresponding mixture, e.g., the corresponding mixture lacks the p53 inhibitor, electroporation enhancer, HDR enhancer and/or cell survival factor. In one embodiment, the homologous recombination efficiency is enhanced at least 20, 30 or 40 fold that of the corresponding mixture, e.g., the corresponding mixture lacks the p53 inhibitor. In one embodiment, the homologous recombination efficiency is enhanced at least 20, 30, 40, 50 or 60 percent or more that of the corresponding mixture, e.g., the corresponding mixture lacks the p53 inhibitor.

In one embodiment, a kit is provided comprising an inhibitor of p53 and one or more of an electroporation enhancer, a cell survival factor or a HDR enhancer. In one embodiment, the inhibitor of p53 comprises a vector for shp53. In one embodiment, the kit further comprises a Cas polypeptide.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1. Single nucleotide substitution targeting design. Schematic diagram illustrates the targeting design for single nucleotide substitution from cytosine (C) to guanine (G) resulting in amino acid change from serine to cysteine by CRISPR. The PAM site denoted by a red box is three nucleotides downstream of the SNP site. The red arrow indicates the double stranded bread induced by Cas9. The repair template also introduces a silent mutation in the PAM site to prevent recutting after editing.

FIG. 2. Schematic for CRISPR protocol design. iPSCs were nucleoporated with either the base protocol, or base protocol +shp53 plasmid, or revised protocol. The major differences of the protocols are illustrated.

FIG. 3. Inhibition of p53 improves HDR efficiency. (A) ICE (Inference of CRISPR Edits) analysis of the genomic DNA derived from nucleoporated iPSC, which shows 2% HDR efficiency. (B) ICE analysis of the genomic DNA derived from nucleoporated iPSC co-transfected with p53 shRNA plasmid, showing improved HDR efficiency to 39%. (C) Addition of p53 inhibition significantly improved the HDR efficiency. (D) The improved HDR efficiency is significantly higher about 20 fold. P values were calculated using Student's t test. Error bars are standard deviation.

FIG. 4. Additional improvement of HDR efficiency. (A) ICE analysis of the genomic DNA derived from nucleoporated iPSC using the revised protocol, shows 65% HDR efficiency. (B) Distribution of the type of edits based on the ICE analysis. (C) The revised protocol significantly improved the HDR efficiency. (D) The improved HDR efficiency is significantly higher about 30 fold compared to the base protocol. P values were calculated using Student's t test. Error bars are standard deviation.

FIG. 5. iPSC cell colonies with pmaxGFP 24-hours post-nucleofection green fluorescent protein (GFP) versus Bright Field at 10X magnification.

FIG. 6. Enhancement of CRISPR editing efficiency in human iPSC. (A) Schematic for CRISPR protocol design. iPSCs were nucleoporated with either the base protocol, or base protocol +shp53 plasmid, or final protocol. The major differences of the protocols are illustrated. (B) Schematic diagram illustrating the targeting design for single nucleotide substitution from cytosine (C) to guanine (G) resulting in amino acid change from serine to cysteine by CRISPR. The PAM site denoted by a red box is three nucleotides from of the SNP site. The red arrow indicates the double-stranded break induced by Cas9. The repair template also introduces a silent mutation in the PAM site to prevent recutting after editing.

FIG. 7. Inhibition of p53 improves HDR efficiency. (A) ICE analysis of the genomic DNA derived from nucleoporated iPSC, which shows 2% HDR efficiency. (B) ICE analysis of the genomic DNA derived from nucleoporated iPSC co-transfected with p53 shRNA plasmid, showing improved HDR efficiency to 39%. (C) Addition of p53 inhibition significantly improved the HDR efficiency. (D) The improved HDR efficiency is significantly higher by about 20 fold. P values were calculated using Student's t test. Error bars are standard deviation.

FIG. 8. Additional improvement of HDR efficiency. (A) ICE analysis of the genomic DNA derived from nucleoporated iPSC using the final protocol, shows 65% HDR efficiency. (B) The final protocol significantly improved the HDR efficiency. (C) The improved HDR efficiency is significantly higher by about 39 fold compared to the base protocol. P values were calculated using Student's t test. Error bars are standard deviation.

FIG. 9. Targeting efficiencies at other sites. (A) Schematic diagram illustrating the targeting design for single nucleotide substitution from guanine (G) to adenine (A) resulting in amino acid change from arginine to glutamine by CRISPR at the rs13045 SNP site. (B) The final protocol significantly improved the HDR efficiency up to 25% or 6 fold. P values were calculated using Student's t test. Error bars are standard deviation.

### DETAILED DESCRIPTION

Genome editing with CRISPR (Clustered Regularly Interspaced Short Palindromic Repeats) is a powerful tool for studying biological functions, which has significantly improved the editing efficiency compared to previous methods such as ZFN (Zinc finger proteins) or TALEN (Transcription activator-like effector nucleases). Pluripotent stem cells can provide an effective method to model disease etiologies. However, they can be difficult to work with, as lower cell survival and editing efficiency continue to be major obstacles in gene editing with iPSC. CRISPR induces apoptosis via p53 activation and leads to poor cell survival. It was hypothesized that mitigation of apoptosis can improve cell survival after CRISPR and editing efficiency. A gene editing protocol for use with stem cells with improved editing efficiency and cell survival, resulting in faster generation of isogenic lines using CRISPR by inhibition of p53 and selection of editing reagents, is described herein.

### The CRISPR/Cas System

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system. The primary products of the CRISPR loci appear to be short RNAs that contain the invader targeting sequences, and are termed guide RNAs

“Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cas1 polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cas1 polypeptide is a Cas1 polypeptide that is a member of CASS3 or CASS7.

In some embodiments, a Cas1 polypeptide is encoded by a nucleotide sequence provided in GenBank at, e.g., GenelD number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA.

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof. RNA Components of CRISPR/Cas

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[n20]GG formula.

### Donors

As noted above, insertion of an exogenous sequence (also called a “donor sequence” or “donor” or “transgene” or “gene of interest”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang, et al. (1987); and Nehls, et al. (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the lysosomal sequences. In other embodiments, the transgene (e.g., with or without additional coding sequences such as for albumin) is integrated into any endogenous locus, for example a safe-harbor locus. See, e.g., U.S. Patent Publication Nos. 2008/0299580; 2008/0159996; and 2010/0218264.

When endogenous sequences (endogenous or part of the transgene) are expressed with the transgene, the endogenous sequences (e.g., albumin, etc.) may be full-length sequences (wild-type or mutant) or partial sequences. Preferably the endogenous sequences are functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

### Exemplary Reagents for the Method

(SEQ ID NO: 2)
sgRNA:
GCUGGAUGACACCAAGGAACGUUUUAGAGCUAGAAAUAGCAAGUUA
AAAUAAGGCUAGUCCGUUAUCAACUUGAAAAAGUGGCACCGAGUCG
GUGCUUUU
(SEQ ID NO: 3)
gRNA:
GCUGGAUGACACCAAGGAACGUUUUAGAGCUAUGCU
(SEQ ID NO: 4)
Unmodified ssODN DNA sequence:
TTAACAGAAAATTCTTACCTCTGGTTTGCTAAGGCTGGATGACACC
AAGCAACCTGATCCCACATCCAAATCCCACTGCTTTTTACCATGAT
TTTCAGGA
Exemplary modified sequence:
/AlT-R-HDR1/T*T*AACAGAAAATTCTTACCTCTGGTTTGCTAA
GGCTGGATGACACCAAGCAACCTGATCCCACATCCAAATCCCACTG
CTTTTTACCATGATTTTCAG*G*A/AlT-R-HDR2/

The star indicates possible sites for modification, e.g., a phosphorothioate modification which renders the internucleotide linkage resistant to nuclease degradation. Phosphorothioate bonds can be introduced between the last 3-5 nucleotides at the 5′- or 3′-end of the oligonucleotide to inhibit exonuclease degradation. Including phosphorothioate bonds throughout the entire oligonucleotide sequence reduces attack by endonucleases as well. Modifications which may be used alone or in combination include but are not limited to phosphorothioate, phosphorodithioate, phophoroselenoate, phosphorodiselenoate, phosphoroanilothioate, phosphoraniladate, phosphoroamidate, and the like. Asymmetric design of the oligonucleotide may improve HDR.

In one embodiment, components of the Alt-R system are employed, which include one or more of an electroporation enhancer, a HDR enhancer, a composition comprising one or more cell survival factors, for use with gRNA or sgRNA, Cas9 enzyme, etc., and optionally one or more oligonucleotides (ssODN). Other reagents may also be employed, e.g., single-stranded, carrier DNA, NATE™ (a highly pure (>95%) blend of innate immune system inhibitors, FBS, or BSA. In one embodiment, fetal serum such as FBS or a protein such as albumin, e.g., BSA, may be added, e.g., to a pulsing buffer.

In one embodiment, electroporation electric field strength is about 325 V/cm to 400 V/cm, e.g., about 375 V/cm. In one embodiment the pulsing duration is about 45 to 120 ms. In one embodiment, plasmid concentrations are at least 0.18 μg/μL to improve transfection efficiency. BSA or FBS in the pulsing buffer. In one embodiment, the pulsing duration is in the range of 30 to 150 milliseconds, e.g., 45 to 120 milliseconds In one embodiment, a HDR enhancer, includes but is not limited to SCR7, RS-1, or Alt-R™ HDR enhancer.

In one embodiment, a cell survival reagent having one or more cell survival factors is employed, e.g., CloneR™, a ROCK inhibitor or Revital Cell (Invitrogen/Life Technologies/ThermoFisher).

The invention will be described by the following non-limiting examples.

### EXAMPLE 1

### Materials and Methods

iPSC Cell Culture Maintenance and Expansion

Induced pluripotent stem cells were derived fibroblasts from patients with normal and those with abnormal SNP variants at the 136, 166 and 704 edit sites on the eIF2AK gene. Fibroblasts were transduced to hiPSC as per the method outlined in Yuan et al. (2018). Cells were maintained in Stemflex Basal Media (Gibco # A334901) with 10% Stemflex Supplement (Gibco # A334901) ‘Stemflex Complete’ in feeder-free conditions on basal matrix of Matrigel (Corning # 47743-706). The cells were dissociated with Accutase (VWR # AT104) for 4-5 minutes, collected in DMEM/F12 (Gibco # A4192001) and centrifuged at 100 g for 5 minutes. Supernatant was then aspirated; cells were plated on to new Matrigel-coated plates with Stemflex media and continued to be incubated at 37° C. in a humidified 5% CO₂ incubator. Cells were not acclimated to single-cell cultures in the passaging prior to nucleofection as CloneR™ supplement (Stem Cell Technologies # 05888) was used. CloneR™ reduces cell death, stress differentiation, and risk of cell culture genetic abnormalities when added to the media post-nucleofection (StemCell Technologies).

### Karyotyping

Karyotyping was performed on the iPSC lines every ten passages or after a new line was established. This was performed by the University of Minnesota Karyotyping services at the Masonic Cancer Genomics Center using the G-banding karyotype technique. Cells were grown as per Yuan lab stem cell culture conditions in a 25 mL cell culture flask (Falcon T25 # 353107) until 60-70% confluent.

### Design of Guide RNA

Guide RNA was designed using Alt-R CRISPR HDR Design Tool from IDT.

### Nucleofection

### Cell Preparation

The lowest passage number available of the cell line was used (<20 passages) when cells were at 80-90% confluency in a 6-well culture plate. The total number of cells for reactions being performed were counted at 1 million cells per reaction. Cell culture media was changed 1-24 hours prior to nucleofection with Stemflex Complete with 1% Revitacell (Gibco # A2644501). A Matrigel-coated 12-well plate was prepared at least 1 hour prior to nucleofection. Stemflex Complete (900 μL), 1% Revitacell (10 μL), 10% (100 μL) CloneR™ and 1% (10 μL) of Alt-R Cas9 HDR Enhancer (IDT # 1081073) was then added to each well to pre-warm in the incubator.

### Component Preparation

Single-Strand Oligo Donor Nucleotides (ssODN) (IDT, custom made) were prepared by resuspending 4 nmol stock to a 100 μM concentration with 40 μL of sterile molecular-grade H₂O (Cytiva # 82007), vortexed to mix, then stored at −20° C.

The crRNA:trcrRNA duplex (gRNA) was prepared by resuspending Alt-R CRISPR-Cas9 crRNA (IDT) in sterile molecular-grade H₂O to a concentration of 200 μM. 5 μL of 200 μM crRNA was combined in a 0.2 mL PCR tube (Sigma Aldrich # BR781316) with 5 μL of 200 μM Alt-R CRISPR-Cas9 trcrRNA-ATTO 550 (IDT # 10810559). The crRNA:trcrRNA duplex was then heated at 95° C. for 5 minutes in a thermocycler and allowed to cool to room temperature before being stored at −20° C. Single guide RNA (sgRNA) (IDT) was diluted to a concentration of 100 μM using sterile molecular-grade H₂O and stored at −20° C.

20 to 30 minutes prior to nucleofection the RNP Complex was prepared by combining 2.1 μL of 1X D-PBS (Gibco # 10010049), 1.2 μL of 100 μM gRNA or sgRNA and 1.7 μL of 10 μg/μL of Alt-R S.p. HiFi Cas9 Nuclease V3 (IDT # 10810559) in a PCR tube and incubated at room temperature for 10 to 20 minutes. If larger amounts of reagents were required, reagent volumes were scaled up such that master mixes could be used.

### Cell Nucleofection Process

Media was aspirated from tissue culture wells. After media removal, 1 mL of 1X D-PBS was added to rinse the cell monolayer and aspirated. Accutase was subsequently added and the cells were incubated for 8 minutes at 37° C. to ensure cells were dissociated as single cells. After incubation, DMEM/F12 was added to neutralize the Accutase. Cells were washed off the plate using a p1000 pipette and transferred to a 15 mL centrifuge tube (Corning # CLS430055). 10 μL of this cell suspension was taken for a cell count. The total amount of cell suspension to provide 1 million cells per reaction was then transferred to a new 15 mL centrifuge tube and centrifuged at 100 g for 5 minutes (for example, 500,000 cells per mL were counted, therefore 2 mL was taken from the cell suspension for one nucleofection reaction). The supernatant was aspirated and 1mL of 1X D-PBS was added to the cell pellet to wash the cell suspension, and then centrifuged again at 100g for 5 minutes. Because RNP components were part of this nucleofection, this wash step was repeated once more to ensure the cell suspension was free of RNase.

The P3 primary cell nucleofector solution was activated prior to use by adding 150 μL of P3 primary cell nucleofector supplement (LONZA # V4XP3032) to 675 μL of the P3 primary cell nucleofector solution (LONZA # V4XP3032) as per iPSC basic protocol for 4D-Nucleofector X-Unit (LONZA, 2010).

The cell pellet was resuspended in 10 μL per reaction of P3 primary cell nucleofector solution (for example, 30 μL of P3 primary cell nucleofector solution was added for a total of three different nucleofection reactions). To each reaction, 1 μL of 0.5 μg/μL pmaxGFP (LONZA # V4XP3032), 1 μL of 100 μM ssODN, 1 μL of 1 μg/μL shp53-f2 plasmid, 0.8 μL of Alt-R Cas9 Electroporation Enhancer (IDT # 1075915), and 5 μL of the pre-prepared RNP complex was combined. P3 primary cell nucleofector solution was added such that the total suspension volume was 20 μL.

A GFP control reaction was created with 10 μL of cells, 1 μL of 0.5 μg/μL pmaxGFP and 9 μL of P3 primary cell nucleofector solution. A no pulse control reaction was created with 10 μL of cells, 1 μL of 0.5 μg/μL pmaxGFP, 1 μL of 100 μM ssODN, 1 μL of 1 μg/μL shp53-f2 plasmid, 0.8 μL of Alt-R Cas9 Electroporation Enhancer, and 5 μL of the pre-prepared RNP complex was combined.

The 20 μL cell suspension and editing components were transferred to a 16-well nucleocuvette strip (LONZA # V4XP3032) and nucleofected using LONZA 4D Nucleofector on the CA137 pulse program. The no pulse control was also transferred to the 16-well nucleocuvette strip, but a pulse program was not assigned to the well. After nucleofection, the nucleocuvette strip was removed from the 4D Nucleofector and incubated at room temperature for 10 minutes without disturbing to allow the lipid-bilayer of the cells to settle/reposition.

After the incubation period, the nucleofected cells were transferred to corresponding wells in the pre-warmed 12-well culture plate using a P20 pipette. They were incubated for 24 hours before the media was changed with Stemflex Complete, 1% Revitacell, and 10% CloneR™ without Alt-R HDR Enhancer. The percentage of iPSCs expressing GFP was calculated after 24 hours. After 48 hours the cells expressing GFP were counted again but the media was not changed unless cells were being transferred to a 6-well plate if over-confluent.

### Determining Homology Directed Repair (HDR) Efficiency

Once cells reached 70-80% confluency they were processed to determine the HDR editing efficiency using Sanger Sequencing (Eurofins). This was done by obtaining a sample of the nucleofected cells to get genomic DNA using the genomic DNA mini prep kit (Zymo # RPI-Z3020). The genomic DNA sample was quantified using a Nanodrop and a PCR reaction was prepared using the primer for the edit site that was nucleofected into the cells. To prepare the sample for sequencing, the PCR product was cleaned using QlAquick PCR clean kit (QIAGEN # 28106) and quantified to send the appropriate amount in 5 μL with 5 μL of the target primer. (forward: 5′ agcccggtttctatgagagc 3′, reverse: 5′ tgggattttgcagcacagat 3′) The results from sequencing were uploaded onto Synthego's ICE analysis software to determine the percentage of HDR, indels and wild type cell populations in each sample.

Isolation and Screening of iPSC Single Clones

Once the percentage of HDR editing was determined, the nucleofected cells were isolated again into single cells by dissociating with Accutase for 8 minutes and approximately 3 million cells (a confluent 6-well plate) were plated into the first 10 cm plate followed by a 1:10 serial dilution into three additional 10 cm plates. 10% CloneR™ was used in the media to maintain single cell health.

Once stem cell colonies had formed after approximately 3-5 days, clones showing homogenous pluripotent morphology, physically isolated from other clones were picked onto a 96-well or 48-well plate depending on the HDR editing efficiency and cell health/growth. These clones were then grown to confluency over approximately another 3-5 days. They were then dissociated using ReleSR (Stem Cell Technologies # 05872) and transferred to larger well plates to allow samples to be taken for genomic DNA to repeat the Sanger Sequencing process to determine if edited clonality had been achieved, or if a second level of subcloning was indicated.

### Screening of 96-Well Plates

Alternatively (if the editing efficiency is low), cells were grown to near confluence on a 96-well plate and screened using real-time qPCR or Sanger Sequencing depending on the edit site being targeted.

At least 1 hour prior to passaging, an identical 96-well plate was set up with Matrigel (this became the duplicate culture plate). Media was aspirated, 30 μL per well of Accutase was added to each well and incubated for 5 minutes at 37° C. 70 μL per well of Stemflex complete with Revitacell media was added to prevent further activity of Accutase. 50 μL of the cell suspension was then taken and added to the duplicate 96-well plate with a further 50 μl of Stemflex complete with Revitacell to make up to 100 μL and continued to be cultured. The remaining cell suspension in each well was then used to screen for potential edited clonal lines.

Genomic DNA was obtained from the cell suspension using QuickExtract DNA Extraction Solution (Lucigen # QE09050) and accompanying protocol (Lucigen, 2018). Following this, the genomic DNA was prepared for real-time PCR using Taqman probes purchased from Appliedbiosystems with the accompanying TaqMan SNP Genotyping qPCR Assay protocol (Appliedbiosystems, 2017). Real-time PCR was performed and analyzed on the Roche LightCycler 490 System using accompanying protocol for relevant SNP probes.

### Results

### Targeting Scheme for Single Nucleotide Substitution

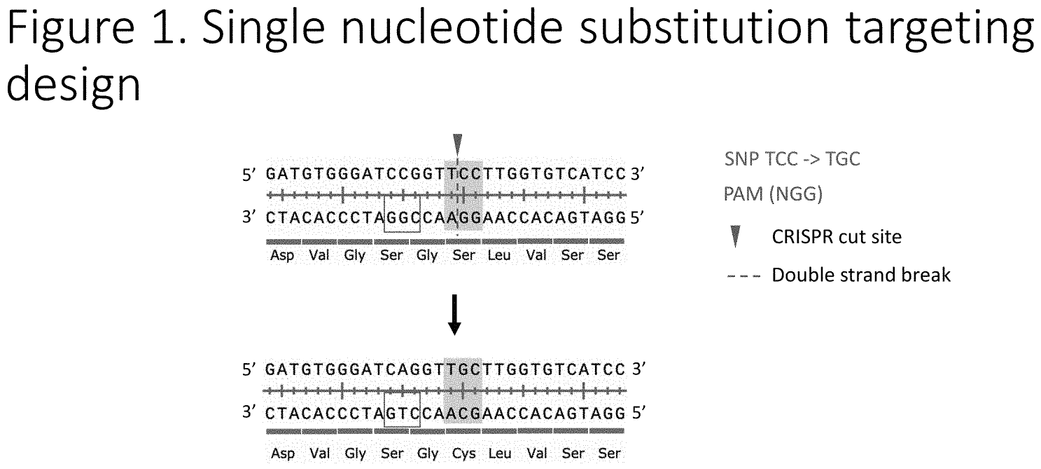

One application for gene editing in iPSC is to produce genetically modified isogenic lines to study how genetic mutations might cause disease. For example, knock-in single nucleotide polymorphism (SNP) variants which were discovered in a genome wide association study for progressive supranuclear palsy, which is a neurodegenerative disease affecting the motor system (Yuan et al., 2018; Hoglinger et al., 2011), were employed to determine HDR efficiency The risk variant rs867529 on the human PERK gene is a single nucleotide polymorphism which encodes a change from cytosine (C) to guanine (G) causing an amino acid substitution from serine to cysteine, located on Chromosome 2. A PAM site four nucleotides from the SNP was identified that directs DNA cut by Cas9 at the SNP. As previously reported, a homozygous HDR event may be more efficiently achieved when the guide RNA induces the cleavage less than ten nucleotides from the intended mutation (Paquet et al., 2016; Kwart et al., 2017). Therefore, by choosing the PAM site less than ten nucleotides from the SNP, the probability for HDR is maximized (FIG. 1).

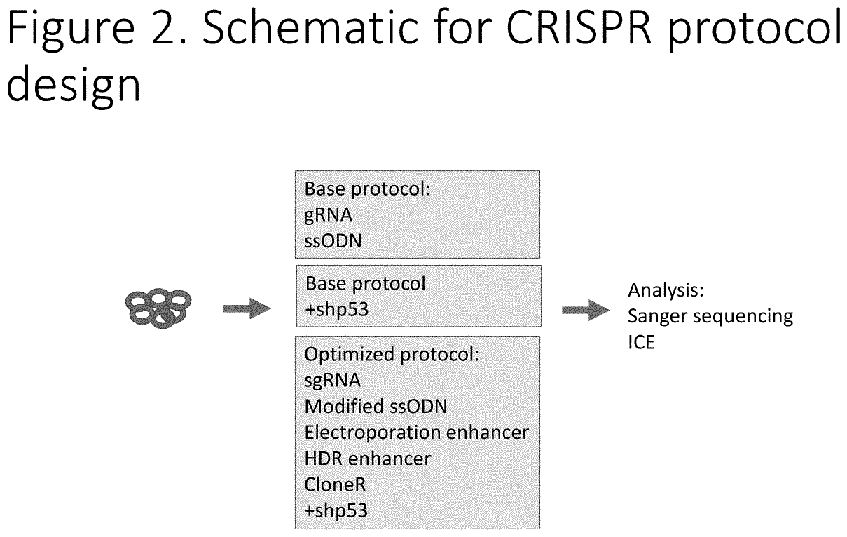

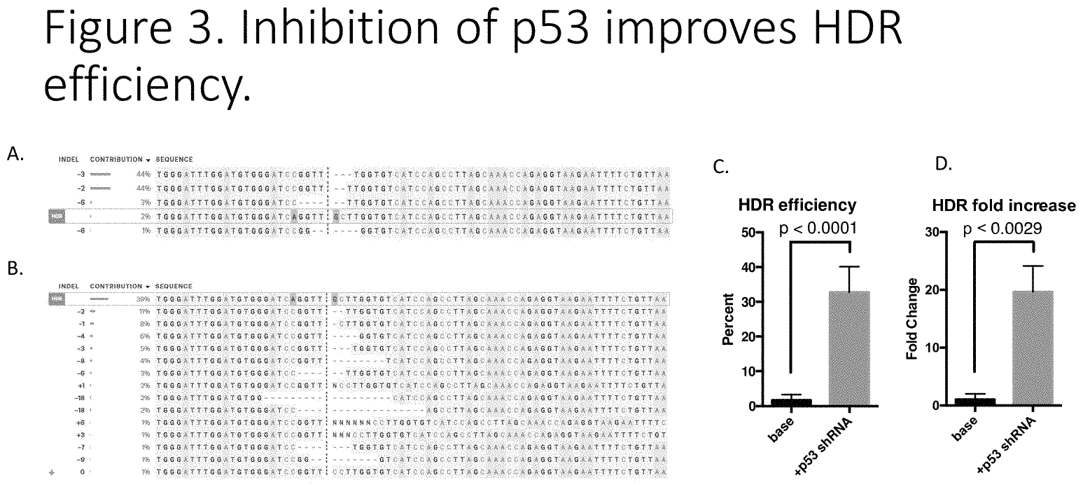

A single strand oligonucleotide (ssODN) was used to introduce the homologous recombination template. A silent mutation was introduced in the PAM site to avoid re-editing (Paquet et al., 2016) thereby improving the editing efficiency. Using Inference of CRISPR Edits (ICE) analysis (Hsiau et al., 2019), an algorithm capable of predicting CRISPR editing based on Sanger sequencing, the targeting efficiency was determined to be around 4% (FIG. 3). However, there was low recovery of the cells, and difficulty IN expanding the cells for subcloning. The reagents used and conditions are listed in FIG. 2.

Improvement of HDR with p53 Knockdown

Previous work indicates that Cas9 causes double stranded break and apoptosis as a cellular response to the DNA damage (Enache et al., 2020; Ihry et al., 2018). It was therefore hypothesized that by improving cell recovery, editing efficiency would be improved. Cells were co-transfected with a p53 shRNA plasmid (Okita et al., 2011). It was found that by transiently inhibiting p53, the HDR rate up improved to 38%, or by 23 fold from base protocol (FIG. 3). However, the cell survival was transient.

### Improvement of HDR by Adding CloneR™

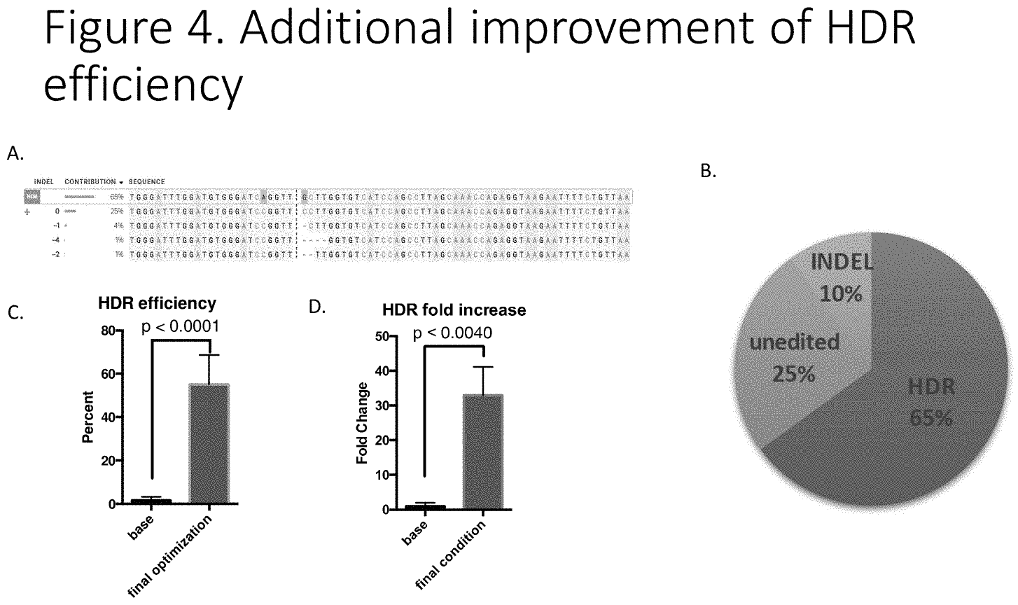

It was hypothesized that addition of an electroporation and HDR enhancer (Integrated DNA Technologies or IDT) and CloneR™ (Stem Cell Technology) would improve HDR and cell survival. It was found that the additional components resulted in improvement of the HDR rate up to 65%, which is 39 times higher compared to the original method (FIG. 4). It was also found that the cells survived subcloning.

### Discussion

A method that shows an improved HDR rate for CRISPR in three different sites in human iPSC is described. It was shown that by improving cell survival and nucleoporation conditions, the HDR can significantly be improved. This protocol is highly efficient, and single clones with genetic edits may be achieved in one month's time. This advance in the methodology makes genetic editing realistically feasible in academic labs, where iPSC is becoming a common tool for studying disease mechanisms and for drug screening. This type of improvement is not limited to one site, but likely to many sites. This method also yields a pool of cells which can be subcloned. The approach does not require drug selection and introduces scarless genomic edits without any markers.

It has been shown that Cas9 causes the cells to commit apoptosis. Therefore, blocking the apoptotic pathway can improve editing efficiency. The present data show that adding p53 shRNA helps to improve the editing efficiency in some sites. It was found that the p53 shRNA effect is limited to the first few days of transduction, which is consistent with the nature of transient transfection. Promotion of cell survival with other supplements such as CloneR™ and ROCK (Rho kinase) inhibition help with survival of the nucleoporated cells and single cell cloning.

The level of improvement in the HDR rate reduces the number of clones to be screened and therefore reduces the overall time for gene editing. The protocol may reduce the time from start to finish to eight weeks, which significantly reduces the amount of time to produce the genetically edited lines.

Previous work shows that sgRNA is superior to crRNA:tracrRNA duplex gRNA (Skarnes et al., 2019). It was found that sgRNA is helpful in the more difficult sites; therefore, using sgRNA improves the HDR rate.

Although cell survival and editing efficiency can be improved by using a certain electroporation device as shown by Kagita et al., a method that employs a p53 inhibitor and CloneR™ does not require a change in electroporation device.

In conclusion, improvements in the Cas9 protein complex and cell survival, can result in genetic editing of a single nucleotide with high HDR efficiency. This protocol allows for the rapid generation of isogenic lines in iPSC.

### EXAMPLE 2

### CRISPR Nucleofection with iPSC Cells Protocol Using a 16-Well Nucleocuvette Strip

### Materials for Cell Culture

TABLE 1

| | |
|---|---|
| Stemflex Basal Media (Gibco | Stored at 4° C. |
| CAT#A334901 450 mL) | |
| Stemflex Supplement (Gibco | Stored at −20° C. Use 5 mL of |
| CAT#A334901 50 mL) | Stemflex Supplement with 45 mL of |
| | Basal Media to make Stemflex Complete |
| | media |
| Revitacell (Gibco | Stored at −20° C. Use 100 μL |
| CAT#A2644501 100X) | aliquot with 10 mL of ‘Stemflex |
| | Complete media + Revitacell’ |
| CloneR ™ (StemCell | Stored at −20° C. Use 1 mL |
| Technologies | aliquot with 9 mL of ‘Stemflex |
| CAT#05888 10 mL) | Complete media + Revitacell’ |
| | to make ‘Cloning Media’ |
| 1X D-PBS (Gibco | Stored at room temperature in tissue |
| CAT#10010049 500 mL) | culture room |
| DMEM/F12 (Gibco | Stored at 4° C. |
| CAT#A4192001 500 mL) | |
| Matrigel (Corning | Stored at −80° C. Use 1 mL |
| CAT#47743-706 5 mL) | aliquot with 11 mL of ‘DMEM/F12’ |
| | to make ‘Matrigel Media’ |
| Accutase (VWR | Stored at −20° C. |
| CAT#AT104 100 mL) | |
| Alt-R Cas9 HDR Enhancer | Stored at −20° C. Use 10 μL |
| (stock solution 3 mM) | per 1 mL of media in culture plates |

Materials for Nucleofection

TABLE 2

| | |
|---|---|
| 16-well Nucleocuvette Strip | Stored at room temperature |
| (LONZA CAT#V4XP3032) | |
| pmaxGFP (LONZA | Stored at 4° C. Dilute to a |
| CAT#V4XP3032 50 uL | 0.5 μg/μL working stock by |
| at 1 μg/μL) | adding 1 uL of ‘P3 Primary |
| | Cell Nucleofector Solution’ |
| | to 1 uL of ‘pmaxGFP’ |
| P3 Primary Cell Nucleofector | Stored at 4° C. |
| Solution (LONZA | |
| CAT#V4XP3032 675 uL) | |
| P3 Primary Cell Nucleofector | Stored at 4° C. Add 150 μL |
| Supplement (LONZA | of ‘P3 Primary Cell Nucleofector |
| CAT#V4XP3032 150 μL) | Supplement’ to the 675 uL of |
| | ‘P3 Primary Cell Nucleofector |
| | Solution’ to ‘activate’ |
| | ‘P3 Primary Cell Nucleofector |
| | Solution’ (can be stored in |
| | 4° C. for <90 days) |
| Alt-R CRISPR-Cas9 crRNA | Stored at −20° C. Dilute |
| (IDT custom made 200 μM) | to a 200 μM working solution |
| | with sterile molecular grade H₂O |
| sgRNA (IDT custom made 100 μM) | Stored at −20° C. |
| Alt-R CRISPR-Cas9 trcrRNA- | Stored at −20° C. |
| ATTO 550 (IDT CAT#1075927 | |
| at 200 μM) | |
| Alt-R S.p. HiFi Cas9 Nuclease | Stored at −20° C. 100 μg |
| V3 (IDT CAT#10810559 500 μg | of Cas9 nuclease = 610 pmol |
| at 10 ug/uL) | |
| Single-Strand Oligo Donor | Stored at −20° C. Stock |
| (ssODN) (IDT comes as 2, | diluted to 100 μM |
| 4 or 10 nmol) | |
| shp53-f2 plasmid or pXLE- | Stored at −20° C. |
| hshP53-F2 plasmid | |
| Alt-R Cas9 Electroporation | Stored at −20° C. |
| Enhancer (IDT CAT#1075915) | |
| Molecular-grade H₂O (Cytiva | This is found in the tissue culture |
| CAT#82007-334 500 mL) | room. Keep sterile |

### Cell Culture Suggestions

Use lowest passage number cells available (about p20)
Replace the media with Stemflex Complete +Revitacell 24 hours before nucleofection or >1-hour prior.
Confluency for iPSC is 80-90% at the time of nucleofection
   ssODN Preparation
1. The exact amount to re-suspend ssODN to a 100 μM concentration can be determined using IDT ‘Resuspension Calculator’https://www.idtdna.com/calc/resuspension/
2. If 4 nmol of ssODN was ordered, add 40 uL of sterile molecular-grade H₂O. If 10 nmol, add 100 μL to get 100 μM working solution
3. Vortex to mix then spin briefly
4. Store at −20° C.
   crRNA:trcrRNA Duplex Preparation (can be made prior to nucleofection and stored at −20° C.)
1. Re-suspend Alt-R CRISPR-Cas9 crRNA or sgRNA in sterile molecular-grade H₂O to a final concentration of 200 μM (for crRNA) or 100 μM (for sgRNA). Use IDT ‘Resuspension Calculator’ https://www.idtdna.com/pages/tools
   This following combination method of crRNA and trcrRNA is not required for sgRNA
2. To make crRNA:trcrRNA duplex gRNA, combine 5 uL of 200 uM Alt-R CRISPR-Cas9 crRNA and 5 μL of 200 μM Alt-R CRISPR-Cas9 trcrRNA-ATTO 550 to make up 10 μL of crRNA:trcrRNA duplex, label tube
   The same trcrRNA-ATTO 550 is used to make all duplexes. Only the Alt-R CRISPR-Cas9 crRNA is dependent upon cell type being nucleofected in this reaction (WT versus MT)
3. This crRNA:trcrRNA Duplex is now 100 μM
4. Heat at 95° C. for 5 minutes using the Thermocycler
5. Remove from heat and allow to cool to room temperature
6. Store at −20° C.

### RNP Complex Preparation (20-30 Minutes Prior to Nucleofection)

1. Per reaction (calculate how much total required), combine:
   2.1 μL 1X D-PBS
   1.2 μL crRNA:tracrRNA (gRNA) (100 uM or 120 pmol) duplex or 1.2 μL of sgRNA (100 μM or 120 pmol)
   1.7 uL of Cas9 enzyme (104 pmol or 64 uM)
   5 uL total per condition/reaction
2. Incubate at room temperature for 10-20 minutes
   Scale up if more RNP complex is necessary
   To save time, prepare RNP complex during the 2×10 minutes centrifugation steps

### Nucleofection Method (for Confluent Wells in a 6-Well Plate of Cells)

1. Prepare a Matrigel 12-well or 24-well plate for the nucleofected cells >1 hour prior
2. After aspirating Matrigel, (for a 12-well plate) add 1 mL of Cloning media (9 mL Stemflex Complete +100 μL Revitacell +1 mL CloneR™) and 10 μL of Alt-R Cas9 HDR Enhancer per 1 mL of media in each well. Incubate. (Half these amounts if using a 24-well plate)
3. Turn on Lonza 4D Nucleofector, select ‘X’, and select the 16-strip

Nucleocuvette with the P3 program ‘CA137’
4. Aspirate media from iPSC cell culture
5. Add 2 mL of 1X D-PBS to clean/rinse cell monolayer, aspirate
6. Add 0.5 mL of Accutase (1 mL for 10 cm plate), incubate at 37° C. for <10 minutes (8 minutes is optimal), check cells have released with microscope
7. Add 2 mL of DMEM/F12 to neutralize the Accutase
8. Gently wash cells off plate using a serological or p1000 pipette and transfer to a 15 mL conical tube
9. Add additional DMEM/F12 to make up to 10 mL
10. Take 10 uL of cell suspension and count using a hemocytometer
   A cell concentration of 1 million cells per reaction (see cell counting protocol)
11. Take total number of cells (per mL of cell suspension) for experiment depending on how many separate reactions are being done
   For example, 3 reactions would need 3 million cells total. If the cell concentration was 0.5 million/mL, 6 mL would need to be taken from the counted suspension to have 3 million cells total.
12. Centrifuge cell suspension @ 100 g for 5 minutes
13. Aspirate and add 1 mL of 1X D-PBS, Centrifuge cell suspension @ 100g for 5 minutes
14. Repeat step 12 to remove potential RNAse when nucleofecting using oligo components
15. Aspirate and re-suspend cells in appropriate μL per reaction of P3 Primary Cell Nucleofector Solution
16. In each centrifuge Eppendorf tube per reaction, add cell suspension & 5 uL pre-prepared RNP complex
If using pmaxGFP (working solution of 0.5 μL), add 1 μL to appropriate reaction tubes
If using ssODN (from stock of 100 μM), add 1 μL to appropriate reaction tubes
If using shp53-f2 plasmid (from stock of 1 μg/μL), add 1 μL to appropriate reaction tubes
Add 0.8 uL of Alt-R Cas9 Electroporation Enhancer (from stock of 100 μM) to appropriate tubes
Add additional P3 Primary Cell Nucleofector Solution if required
17. Pipette suspension up and down 2-3 times to mix and transfer the 20 uL of cell:RNP complex to corresponding 16-well Nucleocuvette strip wells
18. Gently tap the 16-well Nucleocuvette strip on the biohood bench to ensure no air bubbles
19. Place Nucleocuvette strip into the Lonza 4D system and Nucleofect on ‘CA137’ program
20. Check corresponding wells to program. Ensure no pulse control does not have an assigned program. Then select ‘START’. The nucleofector takes only about 30 seconds. Errors will show on the screen
21. Remove Nucleocuvette from nucleofector and incubate at room temperature for 10 minutes
22. After 10 minute room temperature incubation, add 20 μL of the pre-warmed Cloning Media +Alt-R Cas9 HDR Enhancer from the pre-prepared 12-well or 24-well plate to each well in the Nucleocuvette
23. Using a p20 pipette, transfer the 40 μL of suspended cells to corresponding wells in the 12-well or 24-well prepared plate
24. Incubate cells at 37° C., check and change media at 24 hours with Cloning media without Alt-R HDR Enhancer
25. After 48 hours, re-supplement the media with 250 μL per mL of media with CloneR™ (25%). A complete change is not required. This is just to compensate for evaporation/degradation of previous CloneRTM
26. Begin Post-Nucleofection Workflow Protocol after 48 hours

Post-Nucleofection Workflow Protocols

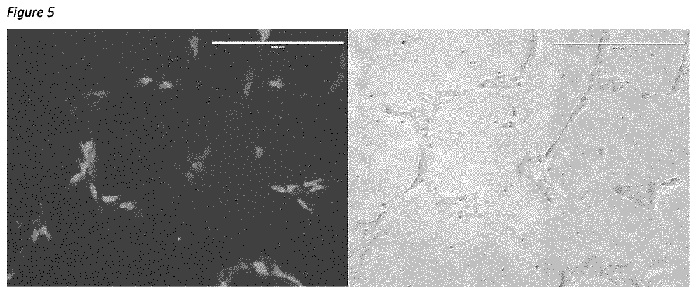

Once cells are nucleofected, the percentage of GFP-expressing cells should be counted at 10x magnification. Take 2 images with the bright field filter and 2 images of the same field of view with the GFP filter per reaction (well). Do this first at 24 hours, then at 48 hours. Save these images onto a USB to count using ImageJ software. After this time, the ability to see GFP under the microscope filter slowly dissipates. See FIG. 5 for an example of GFP-expressing cells versus non-GFP expressing cells. Nucleofected cells should not be harvested for gDNA or split in the first 48 hours as they need time to settle and form healthy colonies.
They should be fed with Cloning media at every change if using Accutase to isolate colonies until stable edited clonal colonies have been established
They should not need splitting during first 48 hours as they have been counted for the appropriate well at time of nucleofection

Once cells reach 70-80% confluency, they are ready to process. There are 3 main pathways to post-nucleofection cell processing. Depending on what stage the cell lines are at (whether they are originally nucleofected, clones or subclones). See following processes:
1) Take cells for genomic DNA send for Sanger Sequencing
2) Take cells for genomic DNA to perform enzyme assay/s
3) Isolate single cells, grow, pick, duplicate plate. Which can then be used for:
   I. qPCR
   II. pick out cells of interest, grow
      III. send to Sanger sequencing
         Taking gDNA from Nucleofected iPSC Cells to Test for Editing using Sanger Sequencing

Materials

TABLE 3

| | |
|---|---|
| Nucleofected cells sample | A sample of about 500 μL from |
| | 2 mL of a fully confluent well in |
| | a 6-well plate. |
| gDNA prep kit (Zymo CAT#RPI- | Stored at room temperature |
| Z3020 500 preps) | |
| Nanodrop | |
| QIAquick PCR clean kit (QIAGEN | Stored at room temperature |
| CAT#28106 250 preps) | |
| Primers | Stored at −20° C. Only use |
| | forward or reverse primer per |
| | sample sent to Sanger Sequencing |
| Molecular-grade H₂O (Cytiva | |
| CAT#82007-334 500 mL) | |
| 1% Agarose gel | |

1) Follow cell harvesting protocol
2) Take half for genomic DNA, then continue to grow or freeze the other half of the cells
   Usually cells should continue to be grown whilst waiting on Sanger Sequencing
3) Follow yellow Zymo genomic DNA preparation kit protocol found with the kit
   Genomic DNA should be re-suspended in 40-50 μL of H₂O, not the elution buffer provided as this may contain EDTA
4) Nanodrop the genomic DNA
5) Follow PCR protocol
6) Run 10 μL of the PCR product on 1% agarose gel to check quality of PCR product
7) Follow Qiagen QlAquick PCR clean up kit protocol found with the kit
   PCR product should be re-suspended in 10 μL of H₂O
   Keep 1^{st} and 2^{nd} eluates if the original amount gDNA was low (<10 ng/μL)
8) Nanodrop eluate to determine correct amount to send to Eurofins for Sanger Sequencing
9) Follow Eurofins Sanger Sequencing Tube Preparation protocol
10) Results from Sanger Sequencing can be analyzed using Synthego's ICE online software and/or SnapGene
11) To analyse using ICE, download the ab.1 file for the desired sample to analyse and the corresponding control file
12) Go to https://ice.synthego.com and drag and drop the ab.1 files into the corresponding ‘control’ and ‘experiment’ sample boxes
13) The guide and ssODN sequences to enter into the ICE program will be found in the ‘Sanger Sequencing’ Box folder (https://umn.app.box.com/folder/101187260115) as an excel spreadsheet file ‘Sequences for Synthego ICE’
14) Select ‘add samples to analysis’ and ‘analyse’
15) These results can be viewed online or downloaded
   Taking genomic DNA from Nucleofected (iPSC or HEK293T) Cells to Test for Editing using Enzyme Assays

### Materials

TABLE 4

| | |
|---|---|
| Nucleofected cells sample | A sample of about 500 μL from |
| | 2 mL of a fully confluent well |
| | in a 6-well plate |
| Genomic DNA prep kit (Zymo | Stored at room temperature |
| CAT#RPI-Z3020 500 preps) | |
| Nanodrop | |
| MspI enzyme (NEB | Stored at −20° C. Keep on |
| CAT#R0106S 5000 units at | ice when in use |
| 20000 units/mL) | |
| Buffer R (NEB CAT#R0153S | Stored at −20° C. Keep on |
| 2000 units at 10000 units/mL) | ice when in use |
| 10X BSA (CAT#R396E 100X) | Stored at −20° C. Stock |
| | solution comes at 100X. Dilute a |
| | working solution t0 10X with |
| | Molecular-grade H₂O. Keep on ice |
| CutSmart Buffer (NEB | Stored at −20° C. Keep on |
| CAT#R0106S 5000 units at | ice when in use |
| 20000 units/mL) | |
| Molecular-grade H₂O (Cytiva | |
| CAT#82007-334 500 mL) | |

1) Follow cell harvesting protocol
2) Take half for genomic DNA, then continue to grow or freeze the other half of the cells
3) Follow yellow Zymo genomic DNA preparation kit protocol
4) Nanodrop gDNA
5) Follow PCR protocol
6) 1% gel agarose preparation
7) See site-specific enzyme preparation and expected results:
   To detect on-target 136 mutations in nucleofected cells using Mspl enzyme
   463 bp+433 bp=no editing/900 bp=editing
   Amount of enzyme required to digest 1 μg of DNA in 1 hour at 37° C. in a volume of 50 μL
   Only use for 136 nucleofected cells
   10 μL 136 PCR
   5.5 μL/6 μL H₂O (+/−tubes)
   2 μL 10× BSA
   2 μL CutSmart (NEB 1.1/2.1/3.1) Buffer
   0.5 μL Mspl enzyme
   Incubate 1 hour at 37° C. (in thermocycler)
   Run on agarose gel for 40 minutes
      Taking genomic DNA from Single (iPSC or HEK293T) Cells to Create Clone/Sub-clones Materials

TABLE 5

| | |
|---|---|
| Nucleofected cells | Check under microscope that there are |
| sample in 6-well plate | clear and defined separate single |
| or 10 cm dish | colonies |
| Cloning Media | 9 mL Stemflex Complete + 1 mL |
| | CloneR ™ + 100 μL Revitacell |
| 1X D-PBS (Gibco | Stored at room temperature in tissue |
| CAT#10010049 500 mL) | culture room |
| DMEM/F12 (Gibco | Stored at 4° C. |
| CAT#A4192001 500 mL) | |
| Blue top cell strainer | Found in the tissue room, bottom |
| tube | drawer underneath the centrifuge |
| Accutase (VWR | Stored at −20° C. 10 mL aliquots |
| CAT#AT104 100 mL) | |
| QuickExtract DNA | Stored at −20° C. 1 mL aliquots |
| Extraction Solution | |
| (Bio search Technologies | |
| CAT#QE9050 50 mL) | |
| Matrigel (Corning | Stored at −80° C. Use 1 mL aliquot |
| CAT#47743-706 5 mL) | with 11 mL of ‘DMEM/F12’ to make |
| | ‘Matrigel Media’ |

1) Once cells are confluent post-nucleofection, they can be split and isolated into single cells
2) Prepare appropriate plates with Matrigel Media for <1 hour and then pre-warm 1×6 well plate or 5×10cm tissue culture dishes with Cloning Media
3) Aspirate media from appropriate plates, add 2 mL of 1X D-PBS, aspirate
4) Add 0.5 mL (for 6-well plate) of Accutase for <10 minutes at 37° C.
5) Add 1 mL of DMEM/F12 to the well to wash and separate
6) Transfer cell suspension to a 15 mL conical flask and top up to 10 mL with DMEM/F12
7) Centrifuge at 100 g for 5 minutes
8) Aspirate supernatant, flick tube to loosen cell pellet
9) Add 2 mL of Cloning Media to cell pellet, pipet to mix
10) Using a p1000 pipette, pre-wet the top of a blue top mesh tube with 500 μL of Cloning Media to allow a medium for cell suspension to flow through the mesh
11) In amounts of 200-500 uL, add the 2 mL of cell suspension onto the mesh lid blue top tube
12) Tap tube on biohood bench to feed cells through mesh
13) Take 10 μL of filtered cell suspension to count cell concentration (see cell counting protocol)
14) Perform a serial dilution by adding the 2 mL of filtered cell suspension from the blue top tube to the 1^{st} well of a 6-well plate [1], take 1 mL of this and add to the 2n^{d} well [2], take 1 mL from [2] and add to well [3], and so on to fill 6 wells of a 6-well plate
15) Check under microscope that cells floating in the dish are in single form
16) Check after 24 hours that cells have adhered and compare each serially diluted plate to see which plate/s to use to pick single colonies. Feed cells.
17) Cells should be ready for picking 48-72 hours after splitting
18) After 48-72 hours when small colonies originating from single cells appear, prepare microscope in the biosafety cabinet
19) Prepare a 48-well or 96-well plate with Matrigel Media <1 hour prior to picking
20) With the appropriate plate under the microscope at 4× magnification, using a p200 pipette on 100 uL setting (to pick to 96-well), pick colonies that are clearly isolated with a good size and shape
21) Allow colonies in each well to form large, good sized colonies (this can take 3-5 days post-picking)
22) Once large colonies have formed in many of the clone wells, prepare/pre-warm a new 96-well plate with 50 μL of Cloning Media in each well
23) Using the multichannel aspirator, aspirate all media
24) Pipette 4 mL of Accutase into a multipipette reservoir and using a p30-300 multipipette, pipette 30 μL of Accutase into all 96 wells
25) Tap plate to ensure the cell dissociation reagent is sitting at the bottom of the wells and not obscured by air bubbles
26) Incubate at 37° C. for <10 minutes (about 5-8 minutes is optimal)
27) Take plate out of the incubator, using a p30-300 multipipette, pipette 70 μL of Cloning Media into all 96 wells of the plate to dilute/stop the Accutase
28) With the multipipette set on 50 μL, one row at a time and changing pipette tips every time, mix wells to ensure cells are loosened and in suspension then transfer 50 μL of cell suspension to the pre-warmed new 96-well plate
29) Transfer this new plate back into the incubator to grow
30) The remaining original plate can now be used to extract genomic DNA and qPCR
31) Follow QuickExtract gDNA protocol to make genomic DNA
32) Follow TaqMan qPCR protocol to perform qPCR on prepared genomic DNA
33) Results from qPCR can be exported as a txt. file to be uploaded into an Excel spreadsheet to be analyzed
34) Depending on qPCR results, the previous 2 protocols can then be carried out from the cells prepared from this protocol

### EXAMPLE 3

TABLE 6
Nucleofection Experiment iPSC example

| | | | |
|---|---|---|---|
| | Test | GFP Only | No Pulse |
| μL | Reaction | Control | Control |

Nucleofection Components

| | | | |
|---|---|---|---|
| ssODN (100 μM) (MT into WT, | 1 | 0 | 1 |
| WT into MT) | | | |
| crRNA: tracrRNA(100 μM)/ | 5 | 0 | 5 |
| Cas9(61 μM) (RNP complex) | | | |
| (WT into WT, MT into MT) | | | |
| GFP (0.5 μg/μL) | 1 | 1 | 1 |
| Alt-R Electroporation | 1 | 1 | 1 |
| Enhancer (100 μM) | | | |
| p53-supressing plasmid | 1 | 1 | 1 |
| (1 μg/μL) | | | |
| Cells (1 million/reaction) | 11 | 11 | 11 |
| (μL can be altered) | | | |
| Additional P3 Primary Cell | 0 | 6 | 0 |
| Nucleofector Solution | | | |
| Total Reaction Volume | 20 | 20 | 20 |

Media Components

| | | | |
|---|---|---|---|
| Alt-R Cas9 HDR Enhancer | 10 | 0 | 10 |
| (in media) | | | |
| CloneR ™ (in media) | Y | Y | Y |
| Revitacell (in media) | Y | Y | Y |

TABLE 7
Alt-R CRISPR-Cas9 crRNA and ssODN

| | crRNA | | | | ssODN | |
|---|---|---|---|---|---|---|
| crRNA | (gRNA) | | ssODN | | Design | |
| (gRNA) | design I.D. | crRNA sequence | Name | ssODN Sequence | number | Notes |
| 136 WT | Hs.Cas9.EIF2AK3.1.AG | GCTGGATGAC | 136 MT #1 | TTAACAGAAA | 263075139/ | To make |
| | | ACCAAGGAAC | HDR | ATTCTTACCT | 317583025 | WT cells |
| | | | template | CTGGTTTGCT | | to 136 |
| | | | | AAGGCTGGAT | | MT cells |
| | | | | GACACCAAGC | | |
| | | | | AACCTGATCC | | |
| | | | | CACATCCAAA | | |
| | | | | TCCCACTGCT | | |
| | | | | TTTTACCATG | | |
| | | | | ATTTTCAGGA | | |
| | | | | (SEQ ID NO: 5) | | |
| 136 MT | Sequence 1 | GCTGGATGAC | 136 WT #1 | TTAACAGAAA | 263075140/ | To make |
| | (ref #262750803) | ACCAAGCAAC | HDR | ATTCTTACCT | 317583026 | MT cells |
| | | | template | CTGGTTTGCT | | to 136 |
| | | | | AAGGCTGGAT | | WT cells |
| | | | | GACACCAAGG | | |
| | | | | AACCTGATCC | | |
| | | | | CACATCCAAA | | |
| | | | | TCCCACTGCT | | |
| | | | | TTTTACCATG | | |
| | | | | ATTTTCAGGA | | |
| | | | | (SEQ ID NO: 6) | | |
| 166 WT | Sequence 1 | TTTCACGGTCT | 166 MT #1 | GTGATTCAAC | 263075142/ | To make |
| | (ref #262750804) | CGGTCCCAC | HDR | TGTGAAAGGA | 317583028 | WT cells |
| | | | template | ACTGTTTCCA | | to 166 |
| | | | | TGCTTTCACG | | MT cells |
| | | | | GTCTTGGTCC | | |
| | | | | CACTGAAAGA | | |
| | | | | GGGCTCCATC | | |
| | | | | CAGGGAAGG | | |
| | | | | AATGATCATC | | |
| | | | | TTATTCCCAAA | | |
| | | | | (SEQ ID NO: 7) | | |
| 166 WT | 166 WT sgRNA | TTTCACGGTCT | 166 MT #2 | TTCAACTGTG | | To make |
| sgRNA | | CGGTCCCAC | HDR | AAAGGAACTG | | WT cells |
| | | | template | TTTCCATGCT | | to 166 |
| | | | | CTCACGGTCT | | MT cells |
| | | | | TGGTCCCATT | | |
| | | | | GGAAGAGGG | | |
| | | | | CTCCATCCAG | | |
| | | | | GGAAGGAATG | | |
| | | | | ATCATCT | | |
| | | | | (SEQ ID NO: 8) | | |
| 166 MT | Hs.Cas9.EIF2AK3.1.AD | TTTCACGGTCT | 166 WT #1 | GTGATTCAAC | 263375429/ | To make |
| | | TGGTCCCAC | HDR | TGTGAAAGGA | 318186204 | MT cells |
| | | | template | ACTGTTTCCA | | to 166 |
| | | | | TGCTTTCACG | | WT cells |
| | | | | GTCTCGGTCC | | |
| | | | | CACTGAAAGA | | |
| | | | | GGGCTCCATC | | |
| | | | | CAGGGAAGG | | |
| | | | | AATGATCATC | | |
| | | | | TTATTCCCAAA | | |
| | | | | (SEQ ID NO: 9) | | |
| 166 MT | 166 MT sgRNA | TTTCACGGTCT | 166 WT #2 | TTCAACTGTG | | To make |
| sgRNA | Hs.Cas9.EIF2AK3.1.AD | TGGTCCCAC | HDR | AAAGGAACTG | | MT cells |
| | | | template | TTTCCATGCT | | to 166 |
| | | | | CTCACGGTCT | | WT cells |
| | | | | CGGTCCCATT | | |
| | | | | GGAAGAGGG | | |
| | | | | CTCCATCCAG | | |
| | | | | GGAAGGAATG | | |
| | | | | ATCATCT | | |
| | | | | (SEQ ID NO: 10) | | |
| 166 | 166 sgRNA | TTCCATGCTTT | 166 WT #3 | TGATTCAACT | | To make |
| sgRNA | (Hs.Cas9.EIF2AK3.1.AL) | CACGGTCT | HDR | GTGAAAGGAA | | MT cells |
| MT/WT | | | template | CTGTCTCCAT | | to 166 |
| | | | | GCTTTCCCGA | | WT cells |
| | | | | TCTCGGTCCC | | |
| | | | | ACTGGAAGAG | | |
| | | | | GGCTCCATCC | | |
| | | | | AGGGAAGGA | | |
| | | | | ATGAT | | |
| | | | | (SEQ ID NO: 11) | | |
| 166 | 166 sgRNA | TTCCATGCTTT | 166 MT #3 | TGATTCAACT | | To make |
| sgRNA | (Hs.Cas9.EIF2AK3.1.AL) | CACGGTCT | HDR | GTGAAAGGAA | | WT cells |
| MT/WT | | | template | CTGTCTCCAT | | to 166 |
| | | | | GCTTTCCCGA | | MT cells |
| | | | | TCTTGGTCCC | | |
| | | | | ACTGGAAGAG | | |
| | | | | GGCTCCATCC | | |
| | | | | AGGGAAGGA | | |
| | | | | ATGAT | | |
| | | | | (SEQ ID NO: 12) | | |
| 704 WT | Sequence 1 | ATATGTTCTTT | 704 MT #1 | CTGCTTCTTTG | 263075144/ | To make |
| | (ref 262750806) | TGTAGAGAA | HDR | TGGTGAAGGA | 317583030 | WT cells |
| | | | template | GCTATGATTT | | to 704 |
| | | | | CAATATGTTC | | MT cells |
| | | | | TTTTGTAGCG | | |
| | | | | AAAGGATCCA | | |
| | | | | TTCTGCGTAT | | |
| | | | | TTTAACTGAT | | |
| | | | | GGTGCATCCA | | |
| | | | | TTGGGCTAG | | |
| | | | | (SEQ ID NO: 13) | | |
| 704 MT | 704 WT | ATATGTTCTTT | 704 WT #1 | CTGCTTCTTTG | 263075143/ | To make |
| | crRNA#l | TGTAGCGAA | HDR | TGGTGAAGGA | 317583029 | MT cells |
| | | | template | GCTATGATTT | | to 704 |
| | | | | CAATATGTTC | | WT cells |
| | | | | TTTTGTAGAG | | |
| | | | | AAAGGATCCA | | |
| | | | | TTCTGCGTAT | | |
| | | | | TTTAACTGAT | | |
| | | | | GGTGCATCCA | | |
| | | | | TTGGGCTAG | | |
| | | | | (SEQ ID NO: 14) | | |
| 704 WT | CD.Cas9.JWBV0826.AB | TTAACTGATG | 704 #4 | tttctcccactt | | To make |
| gRNA #4 | | GTGCATCCAT | MT HDR | ttagCACAGACT | | WT cells |
| | | | ssODN | GGCCACTCAGCT | | to 704 |
| | | | | CTCCTAGCCCA | | MT cells |
| | | | | ATGGATGCAC | | |
| | | | | CATCAGTTAA | | |
| | | | | AATACGCAGA | | |
| | | | | ATGGATCCTT | | |
| | | | | TCgCTACAAA | | |
| | | | | AGAACATATT | | |
| | | | | GAAATCATAG | | |
| | | | | CTCCTTCACC | | |
| | | | | ACAAAGAA | | |
| | | | | (SEQ ID NO: 15) | | |
| 704 WT | CD.Cas9.JWBV0826.AC | ATTCTGCGTAT | 704 #3 MT | cttttagCACAGA | | To make |
| gRNA #2 | | TTTAACTGA | HDR ssODN | CTGGCCACTC | | WT cells |
| | | | | AGCTCTCCTA | | to 704 |
| | | | | GCCCAATGGA | | MT cells |
| | | | | TGCACCATCA | | |
| | | | | GTTAAAATAC | | |
| | | | | GCAGAATGGA | | |
| | | | | TCCTTTCgCTA | | |
| | | | | CAAAAGAACA | | |
| | | | | TATTGAAATC | | |
| | | | | ATAGCTCCTT | | |
| | | | | CACCACAAAG | | |
| | | | | AAGCAG | | |
| | | | | (SEQ ID NO: 16) | | |
| 704 | | ATATGTTCTTT | 704 MT #5 | TGTGGTGAAG | | To make |
| sgRNA | | TGTAGAGAA | HDR ssODN | GAGCTATGAT | | WT cells |
| #1 WT | | | | TTCAATATGT | | to 704 |
| | | | | TCTTTTGTAG | | MT cells |
| | | | | CAAAAGGATC | | |
| | | | | CATTCTGCGT | | |
| | | | | ATTTTAACTG | | |
| | | | | ATGGTGCATCC | | |
| | | | | (SEQ ID NO: 17) | | |
| 704 | | ATATGTTCTTT | 704 WT #3 | TGTGGTGAAG | | To make |
| sgRNA | | TGTAGCGAA | HDR ssODN | GAGCTATGAT | | WT cells |
| #1 MT | | | | TTCAATATGT | | to 704 |
| | | | | TCTTTTGTAG | | MT cells |
| | | | | AAAAAGGATC | | |
| | | | | CATTCTGCGT | | |
| | | | | ATTTTAACTG | | |
| | | | | ATGGTGCATCC | | |
| | | | | (SEQ ID NO: 18) | | |

### EXAMPLE 4

Genome editing using CRISPR (Clustered Regularly Interspaced Short Palindromic Repeats) technology is a powerful tool for studying biological functions, having shown more reliable, editing efficiency than other genome editing methods such as ZFN (Zinc finger proteins) or TALEN (Transcription activator-like effector nucleases). Pairing CRISPR with induced pluripotent stem cells (iPSC) can provide a highly effective method for modelling disease etiologies. However, editing iPSCs with CRISPR has proven difficult, as assuring cell survival and achieving high editing efficiency continue to be major obstacles, with extra variables being introduced depending on the target site. CRISPR can induce apoptosis via p53 activation, leading to poor cell health or death. Mitigation of apoptosis can improve cell survival after CRISPR and thus increase editing efficiency.

As described herein, a protocol is provided to carry out point mutation in iPSCs, increasing the efficiency by inhibition of p53 and selection of editing reagents, resulting in faster generation of isogenic lines using CRISPR. With the addition of p53 inhibition, a 20 fold improvement was observed in the homologous recombination rate. The use of a homologous recombination enhancer to aide with the homologous recombination process and CloneR to enhance cell survival, resulted in improvement of the homologous recombination rate was observed, e.g., by 39 fold or up to 65%. Improved editing efficiency was observed in multiple mutations and cell lines. The protocol can produce clonal cell lines with single genetic editing from start to finish in 8 weeks, which reduces the overall time.

### Introduction

Genome editing is one of the most powerful tools being developed in the field of biotechnology. The ability to manipulate the genome of an organism allows researchers to study complicated evolutionary and medical questions in more complex systems (Robb, 2019). The level of editing varies from theoretically simple gene knockouts to more complicated edits, such as whole gene insertions or point mutations. However, despite its own rapid evolution, current methods of genome editing still suffer from certain difficulties (the history and shortcomings of genome editing is reviewed in (Khalil, 2020; Carroll, 2014; Urnov et al., 2005)). The theory behind modern genome editing is simple: targeted nucleases create double strand breaks (DSB) within genomic DNA at regions of interest, forcing inherent cellular repair mechanisms to fix these breaks. The DSBs increase the likelihood of introducing selected nucleic acids near these sites, which can alter gene function.

There are several existing methods of genome editing, each with their own advantages and shortcomings. Zinc-finger nucleases (ZFN) are designed from a series of bound zinc fingers allowing customization of targeting with a Fold nuclease to create a double-strand break (DSB) (Urnov et al., 2005; Kim et al., 1996; Bibikova et al., 2001; Porteus & Baltimore, 2003). ZFNs have been shown to be adaptable across model organisms, have allowed highly specific targeting, and have proved to be easy to design. However, ZFNs were later found to be less predictable, due to the interaction of the finger domains with adjacent sequencing, sometimes creating off target cleavages and potential cellular toxicity (Gupta et al., 2013). These instances were rare, thus ZFNs are still in use for knockout manipulations, though their use for knockin studies is limited. Transcription activator-like effector nucleases (TALENs) are functionally very similar to ZFNs in that the targeting is specified by engineered proteins bound to a non-specific Fokl restriction endonuclease (Joung & Sander, 2013). TALENs were found to be more specific, coding for single bases rather than three as is the case with ZFNs; however, this increased specificity came at the cost of excess time and money required to produce the large TALENs necessary for such targeting, without a marked change in editing efficiency (Khan, 2019). It was the discovery of the clustered regularly interspaced short palindromic repeats (CRISPR)-Cas systems of prokaryotic organisms (Doudna & Charpentier, 2014), that moved genome editing from a niche community of specialized researchers into the mainstream of life science.

CRISPER technology has become the primary method of genome editing across both plant and animal models (Doudna & Charpentier, 2014). Current methods typically use the nuclease variants Cas9 and Cas12a (Robb, 2019); however, there are a wide range of other CRISPR methods including base editors, transposases, and prime editors, each with their own capabilities and limitations (Anzalone et al., 2020). The CRISPR-Cas9 and -Cas12a systems function by interacting with an RNA guide sequence (gRNA) composed of two segments, a CRISPR RNA (crRNA) which determines the location of genomic targeting and a trans-activating CRISPR RNA (tracrRNA) that directly binds with the Cas nuclease, creating the ribonucleoprotein (RNP) (Jinek et al., 2012). The crRNA and tracrRNA can be combined into a single-guide RNA (sgRNA), with recent findings having shown that use of the combined sgRNA presents an increase in editing efficiency relative to the two part gRNA (Hendel et al., 2015; Basila et al., 2017). Compared to prior editing methods, CRISPR-Cas technology has proved to be simpler to design, providing highly specific targeting and increased editing efficiency, while being cheaper and more usable by a broader community (Wang & Russa, 2016; Li et al., 2018; Zhang 2016). However, despite great advancements in a relatively short amount of time, CRISPR-Cas systems still need troubleshooting.

More recent work has focused on overcoming some of the obstacles currently limiting the efficiency of editing cell lines and target sites. A primary step in genome editing via CRISPR-Cas9 is the dissociation of induced pluripotent stem cells (iPSCs) into single cell culture; however, iPSC culturing of single cells may exhibit increased rates of differentiation or cell death. To prevent death because of dissociation, use of a rho-associated protein kinase (ROCK) inhibitor was found to significantly reduce apoptosis in stem cell cultures (Watanabe et al., 2007; Liu et al., 2012). Cell death has also been linked to the p53 pathway. It was shown that death of nucleofected cells, and thus reduced editing efficiency, is due to activation of the p53 pathway by the DNA double-strand break (DSB) caused by the introduced Cas9 nuclease (Watanabe et al., et al., 2007; Ihry et al., 2018). It stands to reason that inhibition of the p53 pathway would therefore improve editing efficiency by preventing the death of cells that have been edited, or at least undergone a DSB (Conti & Di Micco, 2018).

One application for gene editing in iPSC is to produce genetically modified isogenic lines to study how genetic mutations may cause disease. For example, the genome-wide association study (GWAS) has generated large numbers of single nucleotide polymorphisms (SNP) associated with increased risk of development of diseases. This wealth of information could be further studied using patient derived iPSC in the in-vitro disease-in-a-dish model along with their isogenic control to control the genetic background. However, more than one GWAS risk variant may be associated with a gene of interest, requiring generation of multiple lines with different SNPs, or knocking in the genetic modification sequentially. Therefore, developing a highly efficient and easily adaptable gene editing protocol is essential to allow more labs to generate their own desired genetically edited lines.

A protocol to carry out point mutation in iPSCs, specifically targeting and increasing the efficiency of difficult sites, is provided. Having compiled methods from previous studies to support expansion of existing editing protocols, iPSCs were edited at three sites identified as sites of import for progressive supranuclear palsy (PSP) (Hoglinger et al., 2011; Liu et al., 2013). PSP is a neurodegenerative disease, affecting the motor system, in which a toxic protein, tau, accumulates in the neurons and astrocytes (Ling, 2016). In several large genome-wide association studies (GWAS), the EIF2AK3 (Eukaryotic Translation Initiation Factor 2 Alpha Kinase 3) gene is identified with increased risk in developing PSP (Hoglinger et al., 2011; Kouri et al., 2015; Sanchez-Contreras et al., 2018). This risk variant is in the same linkage disequilibrium with three other single nucleotide polymorphisms (SNP) in the coding region, causing non-synomymous amino acid substitution at p.S136C from a serine to cysteine (rs867529), p.R166Q from a arginine to glutamine (rs13045) and p.S704A from serine to alanine located on Chromosome 2. Patient derived iPSCs have been used to investigate potential disease mechanisms in Progressive Supranuclear Palsy (PSP), a neurodegenerative condition affecting the motor system (Yuan et al., 2018). Using the present protocol, all three sites were edited and homologous recombination efficiency increased by 5-39 fold or up to 65%. This protocol represents an effective method to achieve the development of monoclonal cell lines.

### Materials and Methods

iPSC Cell Culture and Maintenance

Cells were maintained in Stemflex (Gibco # A334901) or mTeSR Plus (STEMCELL Technology # 100-0276) medium in feeder-free conditions on a basal matrix of Matrigel (Corning # 47743-706). ReLeSR (STEMCELL Technologies # 100-0484) was used for routine maintenance splitting. Karyotyping was performed using G-banding by the University of Minnesota Karyotyping services at the Masonic Cancer Genomics Center.

### Nucleofection

Nucleofection was performed when cells were at 80-90% confluency in a 6-well culture plate. Cell culture media was changed 1-24 hours prior to nucleofection with cloning media composed of Stemflex with 1% Revitacell (Gibco # A2644501) and 10% CloneR (STEMCELL Technologies # 05888). Cells were dissociated with Accutase (VWR # AT104) for 4-5 minutes.

The RNP Complex was prepared by combining 0.6 μM guide RNA (IDT) and 0.85 μg/μL of Alt-R S.p. HiFi Cas9 Nuclease V3(IDT#108105559) and incubated at room temperature for 20 to 30 minutes. 0.5 μg pmaxGFP (LONZA # V4XP3032), 5 μM ssODN, and the pre-prepared RNP complex was combined were used in all of the protocols. 50 ng/μL pCXLE-hOCT3/4-shp53-F (Addgene #27077) plasmid was co-transfected with p53 knockdown. In the final protocol, Alt-R Cas9 Electroporation Enhancer at 1:25 dilution (IDT # 1075915) and the pCXLE-hOCT3/4-shp53-F plasmid were also used. In tandem with experimental trials, two external controls were tested simultaneously. A GFP control reaction contained cells and 50 ng/μL pmaxGFP. A no pulse control reaction was created with all components. The 20 μL reaction was transferred to a 16-well nucleocuvette strip (LONZA # V4XP3032) and nucleofected using LONZA 4D Nucleofector on the CA137 pulse program with the exception of the well containing the no pulse control. Following nucleofection, cells were plated on the prepared nucleofection plate and continued to be incubated at 37° C. in a humidified 5% CO₂ incubator. For the final protocol, cells were incubated in media containing 10% CloneR, 1% Revitacell and Alt-R Cas9 HDR Enhancer (IDT #10007910).

### Design of the Guide RNA and the HDR Template

The guide RNA was designed using the IDT Alt-R CRISPR-Cas9 guide RNA (https://www.idtdna.com/site/order/designtool/index/CRISPR_PREDESIGN). Selection of guide RNA was based on the proximity of the cleavage site and the desired gene editing site. Additional criteria include the likelihood of success and the chance of off target editing reported by the program. The following guide RNA sequences were used:

(SEQ ID NO: 20)
rs867529:
GCTGGATGACACCAAGGAAC
(SEQ ID NO: 21)
rs13045:
TTTCACGGTCTCGGTCCCAC
(SEQ ID NO: 22)
rs1805165:
ATATGTTCTTTTGTAGAGAA

The HDR templates were designed using the IDT Alt-R CRISPR HDR Design Tool (https://www.idtdna.com/pages/tools/alt-r-crispr-hdr-design-tool).

The following HDR templates were used:

(SEQ ID NO: 23)
rs867529:
TTAACAGAAAATTCTTACCTCTGGTTTGCTAAGGCTGGATGACA
CCAAGCAACCTGATCCCACATCCAAATCCCACTGCTTTTTACCA
TGATTTTCAGGA
(SEQ ID NO: 24)
rs13045:
GTGATTCAACTGTGAAAGGAACTGTTTCCATGCTTTCACGGTCT
TGGTCCCACTGAAAGAGGGCTCCATCCAGGGAAGGAATGATCAT
CTTATTCCCAAA
(SEQ ID NO: 25)
rsl805165:
CTGCTTCTTTGTGGTGAAGGAGCTATGATTTCAATATGTTCTTT
TGTAGCGAAAGGATCCATTCTGCGTATTTTAACTGATGGTGCAT
CCATTGGGCTAG

### Determining Homology Directed Repair (HDR) Efficiency

Genomic DNA was extracted using the Quick DNA mini prep kit (Zymo Research #3020). The Q5 High-Fidelity DNA polymerase was used to amplify the region around the editing site (New England Biolabs #M0491L) using forward and reverse primers (Table 8). PCR reactions were resolved on a 1% electrophoresis gel and bands of the desired size were excised and purified using a Zymoclean Gel DNA Recovery Kit (Zymo Research #D4001). Samples were then submitted for Sanger sequencing (Eurofins). The results from sequencing were analyzed using Synthego's ICE analysis software to determine the percentage of HDR, INDELs and wild type cell populations in each sample.

TABLE 8.
Primers used for ICE analysis.

| | Forward primer | Reverse primer |
|---|---|---|
| rs867529 | agcccggtttctat | tgggattttgcagc |
| | gagagc | acagat |
| rs13045 | tggcccatggtaaac | cgcttgaacccact |
| | tgagt | tgaacc |
| rs1805165 | tccacgtgaggtacc | GTGGTTGGTCTTGGA |
| | ttgtt | GGAGA |

### Results

### Targeting Scheme for Single Nucleotide Substitution

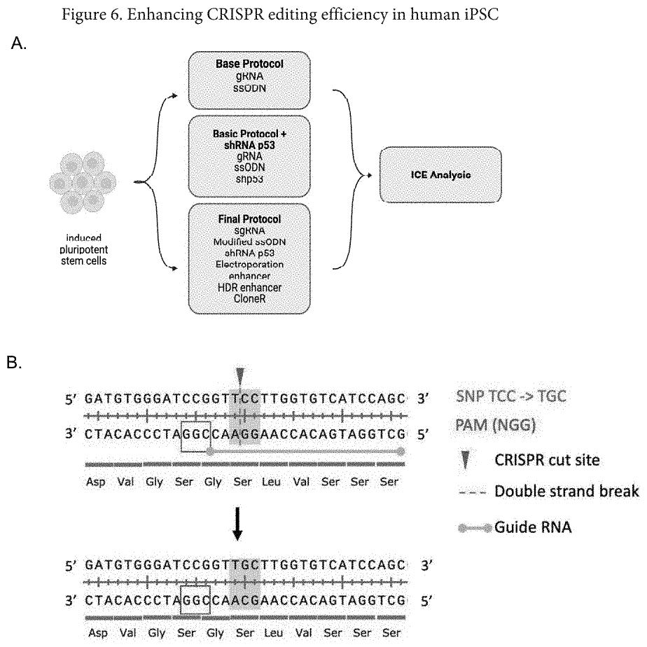

The risk variant rs867529 on the human PERK gene is a SNP encoding a single nucleotide substitution from cytosine (C) to guanine (G) resulting in an amino acid change from serine to cysteine at amino acid position 136 (FIG. 6B). We identified a PAM site (protospacer adjacent motif) four nucleotides from the SNP and directed a DNA cut by Cas9 at the SNP. As previously reported, a homozygous homology directed recombination (HDR) event is best achieved when the guide RNA induces the cleavage less than ten nucleotides from the intended mutation (Urnov et al., 2005; Kim et al., 1996). Therefore, by choosing the PAM site less than ten nucleotides from the SNP, the probability for HDR to occur was enhanced (FIG. 6B).

Single strand oligonucleotide (ssODN) was used as the repair template to introduce the desired editing for the homologous recombination. In the template, a silent mutation was introduced in the PAM site to avoid re-editing (Urnov et al., 2005), thereby improving the editing efficiency. Using Inference of CRISPR Edits (ICE) analysis, we determined the targeting efficiency to be an average of 1.6% (FIG. 6A). However, there was low recovery of the cells, and difficulty in expanding the cells for subcloning.

### Improvement of HDR

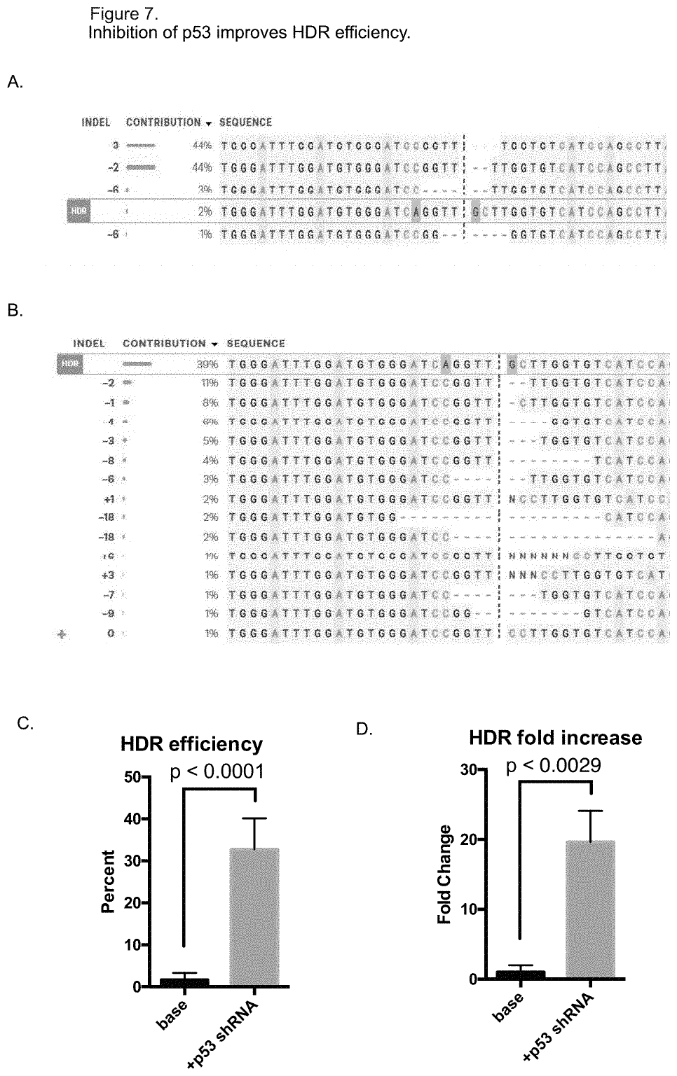

Previous work indicates that Cas9 causes a double stranded break and apoptosis via p53 activation as a cellular response to the DNA damage (Porteus & Baltimore, 2003; Anzalone et al., 2020). It was hypothesized that if p53 activation was inhibited, cell recovery would be improved, which in turn would improve editing efficiency. Upon co-transfection of a plasmid encoding shRNA against p53 (Jinek et al., 2012), the HDR rate increased to an average of 33%, which is 20 times higher than it was from the base protocol (FIG. 7C, 7D). We found that although we had more cells that survived immediately after nucleoporation, the iPSCs had a tendency to die a few days later. It was hypothesized that the p53 shRNA plasmid expression is transient.

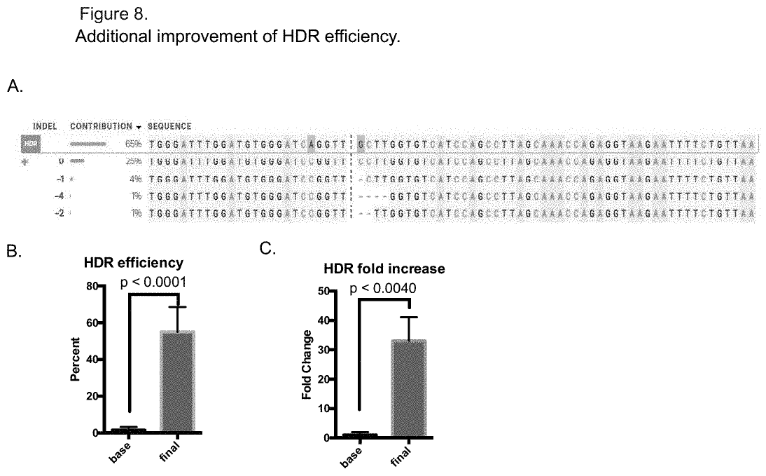

An electroporation and HDR enhancer (IDT) was added to improve the HDR, and CloneR (Stem Cell Technology) was added to improve cell survival. When these additional reagents were applied, the HDR increased to 65%, which is 33 times higher than the base protocol (FIG. 8A, 8B). The nucleoporated cells not only survived, but also could be expanded for subcloning.

Targeting Efficiency at Other Sites and iPSC Lines

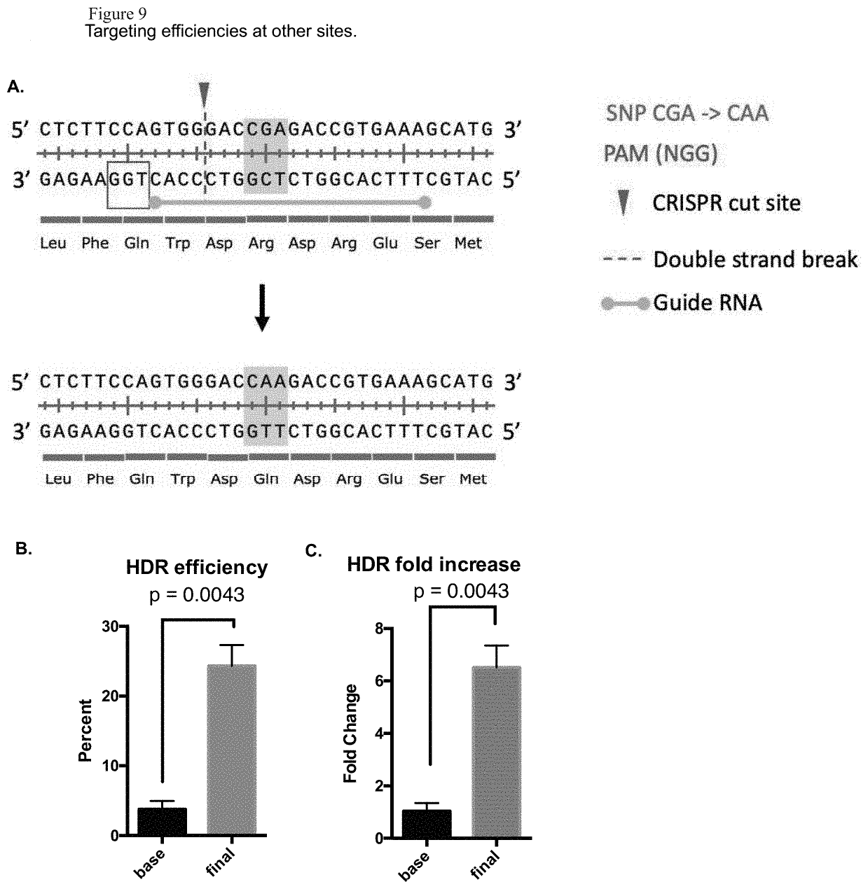

It was hypothesized that the above modified protocol could be applied to genomic editing at other sites. Another SNP variant (rs13045) for the EIF2AK3 gene was tested, which changes the nucleotide at amino acid position 166 from guanine (G) to adenine (A), resulting in amino acid change from arginine (R) to glutamine (Q) in exon 3 of the PERK gene (FIG. 9A). A Cas9 cleavage site which is four nucleotides away from the SNP was tested. Using the base protocol, the HDR efficiency was about 4% (FIG. 9B). When combined, the HDR efficiency was increased to about 25% (FIG. 9B). The increase in HDR efficiency is about 5-fold higher compared to the base protocol (FIG. 9C).

Another SNP variant (in the PERK gene associated with tauopathy) was tested. At this SNP (rs1805165), thymine (T) is substituted to guanine (G) causing an amino acid substitution from serine (S) to alanine (A). When the base protocol was used, HDR could not be detected. However, when the combined protocol was used, up to 1-5% HDR efficiency was observed depending on the donor template (Table 8). The combined protocol was used to correct the risk variant in rs1805165 in the iPSC line derived from a patient PSP1.2 (Yuan et al., 2018) from G to T. The highest editing efficiency was 24% with homologous recombination at this site (Table 9).

TABLE 9
HDR efficiency at different loci and iPSC lines.

| | single | | Highest | Highest |
|---|---|---|---|---|
| | nuclear | | HDR rate | HDR rate |
| locus | substitution | iPSC line | base protocol | final protocol |
| rs867529 | C > G | NDC1.1 | 2% | 65% |
| rs13045 | G > A | NDC1.1 | 4% | 25% |
| rs1805165 | T > G | NDC1.1 | 0% | 5% |
| rs1805165 | G > T | PSP1.2 | 0% | 24% |

### Discussion

An improved HDR rate for CRISPR editing in three different genetic loci in human iPSC is shown. With p53 inhibition and nucleoporation conditions, we the HDR and editing efficiency is improved, even for challenging sites that did not respond to the previous method. This combined protocol is highly efficient, and single clones with genetic edits may be achieved in a truncated time. This advance in the methodology makes genetic editing realistically feasible in academic labs, where iPS cells are becoming a common tool for studying disease mechanisms and for drug screening. This type of improvement is not limited to one site, but likely applies to many sites and different cell lines without any selection. This method yields a pool of cells which can be subcloned.

It has been shown that Cas9 causes the cells to commit apoptosis; thus, blocking the apoptotic pathway can improve editing efficiency. In addition, the electroporation process is damaging to cells and can induce cell death. The present data shows that adding p53 shRNA helps to improve the editing efficiency in some sites, though it may not apply to all cases. The combination of the p53 shRNA and promotion of cell survival with other supplements such as CloneR and ROCK inhibition helps with the editing efficiency, likely due to survival of the nucleoporated cells. One protocol from another lab also showed this inhibition of the p53 pathway with co-transfection with a plasmid encoding BCL-XL, which is the anti-apoptotic isoform of the BCL2-like 1 (BCL2L1) gene (Li et al., 2018). Although this protocol showed improvement in HDR, it also utilized drug selection. In contrast, the present protocol does not require drug selection. A protocol from another lab showed that by using a different electroporation device, they were able to improve the cell survival and editing efficiency (Kagita et al., 2021). This would require acquisition of a device, which can be expensive. Because the present protocol uses the Amaxa nucleofector, it can be adapted in many labs which already have the Amaxa nucleofector.

Traditionally, a single anti-apoptotic agent is used to improve cell survival in iPSCs. Because both the electroporation and DSB of chromatin induced by Cas9 are damaging to the cells, multiple anti-apoptotic agents may be necessary. Recently, a cocktail of 4 different compounds has been shown to improve iPSC survival (Chen et al., 2021). It was found that by using multiple pro-survival methods, the chance of getting the desired gene-edited clone increased. Utilization of anti-apoptotic agents may raise concerns about promoting chromosome abnormality for growth advantage. Karyotyping with G-banding was performed on the genetic edited single clones. It was found that the clones have normal diploid karyotype. The present data suggests that temporary usage of the anti-apoptotic agents does not promote the selection of an abnormal karyotype.

Although the editing efficiency for each genetic locus can vary due to a variety of conditions, such as chromatin accessibility and presence of a G/C rich region, improvement in cell survival can increase the chance of obtaining the genetically edited clone. Other changes can help to increase the editing efficiency, e.g., by improving the specificity of the guide RNA and/or increasing the chromatin accessibility, so that challenging sites can be successfully targeted. The level of improvement in the HDR rate reduces the number of clones to be screened. The present protocol can produce clonal cell lines with genetic edits from start to finish in 8 weeks, which significantly reduces the overall time required. In conclusion, CRISPR editing efficiency can be improved by increasing cell survival and selection of editing reagents. This protocol can allow for the rapid generation of isogenic lines in iPSC.

### REFERENCES

IDT. (revised 2019). Alt-R CRISPR-Cas9 System.
https://umn.box.com/s/pb36cqx845r5craf2asqmp75o0gw927t\
Budde et al., (2017). Precision genome-editing with CRISPR/Cas9 in human induced pluripotent stem cells. Not peer reviewed.
http://dx.doi.org/10.1101/187377.
Anzalone et al., Nature Biot., 38:824 (2020).
Basila et al., PLOS ONE, 12:e0188593 (2017).
Bibikova et al., Mol. Cell. Biol., 21:289 (2001).
Budde et al., bioRxiv, 187377 (2017).
Carroll, Annu. Rev. Biochem., 83:409 (2014).
Chen et al., Nature Meth., 18:528 (2021).
Conti & Di Micco, Genome Med., 10:66 (2018).
Doudna & Charpentier, Science, 346:1258096 (2014).
Enache et al., Nature Genetics, 52:662 (2020).
Gupta et al., Genome Res., 23:1008 (2013).
Hendel et al., Nat. Biot., 33:985 (2015).
Hoglinger et al., Nat. Genet., 43:699 (2011).
Hsiau et al., bioRxiv, 251082 (2019).
Ihry et al., Nat. Med., 24:939 (2018).
Jinek et al., Science, 337:816 (2012).
Joung & Sander, Nat. Rev. Mol. Cell Biol., 14:49 (2013).
Kagita et al., Stem Cell Rep., 16:985 (2021).
Khalil, J. Gen. Eng. Biot., 18:68 (2020).
Khan, Mol. Ther. Nucleic Acids, 16:326 (2019).
Kim et al., PNAS, 93:1156 (1996).
Kouri et al., Nat. Commun., 6:7247 (2015).
Kwart et al., Nature Protocols, 12:329 (2017).
Li et al., Nucleic Acids Res., 46:10195 (2018).
Ling, J. Mov. Disord., 9:3 (2016).
Liu et al., Am. J. Pathol., 180:599 (2012).
Liu et al., Neurobiol. Aging, 34:1519 (2013).
Okita et al., Nat. Methods, 8:409 (2011).
Paquet et al., Nature, 533:125 (2016).
Porteus & Baltimore, Science, 300:763 (2003).
Richardson et al., Nature Biotech., 34:339 (2016).
Robb, Curr. Proto. Essen. Lab. Tech., 19:e36 (2019).
Sanchez-Contreras et al., Mol. Neurod., 13:37 (2018).
Skarnes et al., Methods, 164, :18 (2019).
Urnov et al., Nature, 435:646 (2005).
Wang et al., Ann. Rev. Biochem., 85:227 (2016).
Watanabe et al., Nat. Biotech., 25:681 (2007).
Yuan et al., Hum. Mol. Genet., 27:3951 (2018).
Zhang et al., Bioengineered, 7:166 (2016).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to enhance homology directed recombination (HDR) efficiency, comprising:
a) combining mammalian cells, an electroporation enhancer, a ribonucleoprotein complex comprising gRNA or sgRNA for a selected target in the genome of the cells and Cas polypeptide, an inhibitor of p53, and a single stranded oligonucleotide donor template comprising one or more homology arms for the selected target, thereby providing a mixture;
b) subjecting the mixture to electroporation conditions; and
c) incubating the electroporated cells with a HDR enhancer and a composition comprising one or more cell survival factors for a period of time so as to provide for enhanced homologous recombination at the target in the electroporated cells relative to a corresponding mixture that lacks one or more of the electroporation enhancer, the inhibitor of p53, the HDR enhancer or one or more of the cell survival factors.

2. The method of claim 1 further comprising incubating the cells of c) in a medium that includes one or more cell survival factors but not a HDR enhancer.

3. The method of claim 1 wherein the cells are stem cells.

4. The method of claim 3 wherein the cells are pluripotent stem cells.

5. The method of claim 4 wherein the cells are induced pluripotent stem cells.

6. The method of claim 1 wherein the cells are human cells.

7. The method of claim 1 wherein the electroporation enhancer comprises nucleic acid or NATE™.

8. The method of claim 7 wherein the electroporation enhancer comprises TTAGCTCTGTTTACGTCCCAGCGGGCATGAGAGTAACA AGAGGGTGTGGTAATATTACGGTACCGAGCACTATCGA TACAATATGTGTCATACGGACACG (SEQ ID NO:1), or a nucleic acid sequence having at least 80%, 82%, 84%, 85%, 86%, 88%, 90%, 92%, 94%, 95%, 96%, 97%, 98% or 99% nucleotide sequence identity thereto.

9. The method of claim 1 wherein the homology arms of the single stranded oligonucleotide are about 30 to about 50 nucleotides in length.

10. The method of claim 1 wherein the composition comprising the one or more cell survival factors comprises CloneR™, ROCK inhibitor, Revital Cell Supplement, Y-27632 or fasudil.

11. The method of claim 1 wherein the composition comprises one or more of chroman 1, emricasan, polyamines, or trans-ISRIB.

12. The method of claim 1 further comprising cloning the electroporated cells.

13. The method of claim 1 wherein the HDR enhancer comprises SCR7, RS-1, L755507, ABT-751, nocodazole, brefeldin A, 3′-azido-3′deoxythymidie, (Z)-4-hydroxytamoxifen or Alt-RTM HDR enhancer, NU7026, Trichostatin A, MLN4924, NSC 19630, NSC 15520, AICAR, Resveratrol, STL127685, B02, valproic acid, L755507, XL413, mimosin, thymidine, hydroxy urea, aphidicolin, VE-822, M3814, KU-0060648, NU7441, i53, B02, inhibition of Ku70/Ku80, overexpression of RAD51, or inhibition of DNA ligase IV.

14. The method of claim 1 wherein homologous recombination efficiency is enhanced by at least 5 times that of the corresponding mixture.

15. The method of claim 14 wherein the corresponding mixture lacks a vector for p53 inhibition.

16. The method of claim 1 wherein the period of time for c) is from about 2 hours to about 36 hours.

17. A kit comprising an inhibitor of p53 and one or more of an electroporation enhancer, a composition comprising one or more cell survival factors or a HDR enhancer.

18. The kit of claim 17 wherein the inhibitor of p53 comprises a vector that expresses shp53.

19. The kit of claim 17 further comprising a Cas polypeptide.

20. The kit of claim 17 which comprises the electroporation enhancer, the composition comprising the one or more cell survival factors and the HDR enhancer


SEQUENCE LISTING (94 sequences)

SEQ ID NO: 1 - DNA, 100 bp, synthetic construct
  source 1..100: mol_type = other DNA
ttagctctgtttacgtcccagcgggcatgagagtaacaagagggtgtggtaatattacggtaccgagcactatcgatacaatatgtgtcatacggacacg

SEQ ID NO: 2 - RNA, 99 nt, synthetic construct
  source 1..99: mol_type = other RNA
gctggatgacaccaaggaacgtttagagctagaaatagcaagttaaaataaggctagtccgttatcaacttgaaaaagtggcaccgagtcggtgctttt

SEQ ID NO: 3 - RNA, 35 nt, synthetic construct
  source 1..35: mol_type = other RNA
gctggatgacaccaaggaacgtttagagctatgct

SEQ ID NO: 4 - DNA, 100 bp, synthetic construct
  source 1..100: mol_type = other DNA
ttaacagaaaattcttacctctggtttgctaaggctggatgacaccaagcaacctgatcccacatccaaatcccactgctttttaccatgattttcagga

SEQ ID NO: 5 - DNA, 100 bp, synthetic construct; identical to SEQ ID NO: 4
  source 1..100: mol_type = other DNA

SEQ ID NO: 6 - DNA, 100 bp, synthetic construct
  source 1..100: mol_type = other DNA
ttaacagaaaattcttacctctggtttgctaaggctggatgacaccaaggaacctgatcccacatccaaatcccactgctttttaccatgattttcagga

SEQ ID NO: 7 - DNA, 100 bp, synthetic construct
  source 1..100: mol_type = other DNA
gtgattcaactgtgaaaggaactgtttccatgctttcacggtcttggtcccactgaaagagggctccatccagggaaggaatgatcatcttattcccaaa

SEQ ID NO: 8 - DNA, 86 bp, synthetic construct
  source 1..86: mol_type = other DNA
ttcaactgtgaaaggaactgtttccatgctctcacggtcttggtcccattggaagagggctccatccagggaaggaatgatcatct

SEQ ID NO: 9 - DNA, 100 bp, synthetic construct
  source 1..100: mol_type = other DNA
gtgattcaactgtgaaaggaactgtttccatgctttcacggtctcggtcccactgaaagagggctccatccagggaaggaatgatcatcttattcccaaa

SEQ ID NO: 10 - DNA, 86 bp, synthetic construct
  source 1..86: mol_type = other DNA
ttcaactgtgaaaggaactgtttccatgctctcacggtctcggtcccattggaagagggctccatccagggaaggaatgatcatct

SEQ ID NO: 11 - DNA, 84 bp, synthetic construct
  source 1..84: mol_type = other DNA
tgattcaactgtgaaaggaactgtctccatgctttcccgatctcggtcccactggaagagggctccatccagggaaggaatgat

SEQ ID NO: 12 - DNA, 84 bp, synthetic construct
  source 1..84: mol_type = other DNA
tgattcaactgtgaaaggaactgtctccatgctttcccgatcttggtcccactggaagagggctccatccagggaaggaatgat

SEQ ID NO: 13 - DNA, 100 bp, synthetic construct
  source 1..100: mol_type = other DNA
ctgcttctttgtggtgaaggagctatgatttcaatatgttcttttgtagcgaaaggatccattctgcgtattttaactgatggtgcatccattgggctag

SEQ ID NO: 14 - DNA, 100 bp, synthetic construct
  source 1..100: mol_type = other DNA
ctgcttctttgtggtgaaggagctatgatttcaatatgttcttttgtagagaaaggatccattctgcgtattttaactgatggtgcatccattgggctag

SEQ ID NO: 15 - DNA, 135 bp, synthetic construct
  source 1..135: mol_type = other DNA
tttctcccacttttagcacagactggccactcagctctcctagcccaatggatgcaccatcagttaaaatacgcagaatggatcctttcgctacaaaagaacatattgaaatcatagctccttcaccacaaagaa

SEQ ID NO: 16 - DNA, 130 bp, synthetic construct
  source 1..130: mol_type = other DNA
cttttagcacagactggccactcagctctcctagcccaatggatgcaccatcagttaaaatacgcagaatggatcctttcgctacaaaagaacatattgaaatcatagctccttcaccacaaagaagcag

SEQ ID NO: 17 - DNA, 81 bp, synthetic construct
  source 1..81: mol_type = other DNA
tgtggtgaaggagctatgatttcaatatgttcttttgtagcaaaaggatccattctgcgtattttaactgatggtgcatcc

SEQ ID NO: 18 - DNA, 81 bp, synthetic construct
  source 1..81: mol_type = other DNA
tgtggtgaaggagctatgatttcaatatgttcttttgtagaaaaaggatccattctgcgtattttaactgatggtgcatcc

SEQ ID NO: 19 - skipped

SEQ ID NO: 20 - DNA, 20 bp, synthetic construct
  source 1..20: mol_type = other DNA
gctggatgacaccaaggaac

SEQ ID NO: 21 - DNA, 20 bp, synthetic construct
  source 1..20: mol_type = other DNA
tttcacggtctcggtcccac

SEQ ID NO: 22 - DNA, 20 bp, synthetic construct
  source 1..20: mol_type = other DNA
atatgttcttttgtagagaa

SEQ ID NO: 23 - DNA, 100 bp, synthetic construct; identical to SEQ ID NO: 4
  source 1..100: mol_type = other DNA

SEQ ID NO: 24 - DNA, 100 bp, synthetic construct; identical to SEQ ID NO: 7
  source 1..100: mol_type = other DNA

SEQ ID NO: 25 - DNA, 100 bp, synthetic construct; identical to SEQ ID NO: 13
  source 1..100: mol_type = other DNA

SEQ ID NO: 26 - DNA, 20 bp, synthetic construct
  source 1..20: mol_type = other DNA
gctggatgacaccaaggaac

SEQ ID NO: 27 - DNA, 20 bp, synthetic construct
  source 1..20: mol_type = other DNA
gctggatgacaccaagcaac

SEQ ID NO: 28 - DNA, 20 bp, synthetic construct
  source 1..20: mol_type = other DNA
tttcacggtctcggtcccac

SEQ ID NO: 29 - DNA, 20 bp, synthetic construct
  source 1..20: mol_type = other DNA
tttcacggtcttggtcccac

SEQ ID NO: 30 - DNA, 19 bp, synthetic construct
  source 1..19: mol_type = other DNA
ttccatgctttcacggtct

SEQ ID NO: 31 - DNA, 20 bp, synthetic construct
  source 1..20: mol_type = other DNA
atatgttcttttgtagagaa

SEQ ID NO: 32 - DNA, 20 bp, synthetic construct
  source 1..20: mol_type = other DNA
atatgttcttttgtagcgaa

SEQ ID NO: 33 - DNA, 20 bp, synthetic construct
  source 1..20: mol_type = other DNA
ttaactgatggtgcatccat

SEQ ID NO: 34 - DNA, 20 bp, synthetic construct
  source 1..20: mol_type = other DNA
attctgcgtattttaactga

SEQ ID NO: 35 - DNA, 20 bp, synthetic construct
  source 1..20: mol_type = other DNA
atatgttcttttgtagagaa

SEQ ID NO: 36 - DNA, 20 bp, synthetic construct
  source 1..20: mol_type = other DNA
atatgttcttttgtagcgaa

SEQ ID NO: 37 - DNA, 20 bp, synthetic construct
  source 1..20: mol_type = other DNA
agcccggtttctatgagagc

SEQ ID NO: 38 - DNA, 20 bp, synthetic construct
  source 1..20: mol_type = other DNA
tgggattttgcagcacagat

SEQ ID NO: 39 - DNA, 20 bp, synthetic construct
  source 1..20: mol_type = other DNA
tggcccatggtaaactgagt

SEQ ID NO: 40 - DNA, 20 bp, synthetic construct
  source 1..20: mol_type = other DNA
cgcttgaacccacttgaacc

SEQ ID NO: 41 - DNA, 20 bp, synthetic construct
  source 1..20: mol_type = other DNA
tccacgtgaggtaccttgtt

SEQ ID NO: 42 - DNA, 20 bp, synthetic construct
  source 1..20: mol_type = other DNA
gtggttggtcttggaggaga

SEQ ID NO: 43 - DNA, 33 bp, synthetic construct
  source 1..33: mol_type = other DNA
gatgtgggatccggttccttggtgtcatccagc

SEQ ID NO: 44 - DNA, 33 bp, synthetic construct
  source 1..33: mol_type = other DNA
gctggatgacaccaaggaaccggatcccacatc

SEQ ID NO: 45 - DNA, 33 bp, synthetic construct
  source 1..33: mol_type = other DNA
gatgtgggatccggttgcttggtgtcatccagc

SEQ ID NO: 46 - DNA, 33 bp, synthetic construct
  source 1..33: mol_type = other DNA
gctggatgacaccaagcaaccggatcccacctc

SEQ ID NO: 47 - DNA, 72 bp, synthetic construct
  source 1..72: mol_type = other DNA
tgggatttggatgtgggatccggtttggtgtcatccagccttagcaaaccagaggtaagaattttctgttaa

SEQ ID NO: 48 - DNA, 73 bp, synthetic construct
  source 1..73: mol_type = other DNA
tgggatttggatgtgggatccggttttggtgtcatccagccttagcaaaccagaggtaagaattttctgttaa

SEQ ID NO: 49 - DNA, 69 bp, synthetic construct
  source 1..69: mol_type = other DNA
tgggatttggatgtgggatccttggtgtcatccagccttagcaaaccagaggtaagaattttctgttaa

SEQ ID NO: 50 - DNA, 75 bp, synthetic construct
  source 1..75: mol_type = other DNA
tgggatttggatgtgggatcaggttgcttggtgtcatccagccttagcaaaccagaggtaagaattttctgttaa

SEQ ID NO: 51 - DNA, 69 bp, synthetic construct
  source 1..69: mol_type = other DNA
tgggatttggatgtgggatccggggtgtcatccagccttagcaaaccagaggtaagaattttctgttaa

SEQ ID NO: 52 - DNA, 75 bp, synthetic construct; identical to SEQ ID NO: 50
  source 1..75: mol_type = other DNA

SEQ ID NO: 53 - DNA, 72 bp, synthetic construct; identical to SEQ ID NO: 47
  source 1..72: mol_type = other DNA

SEQ ID NO: 54 - DNA, 73 bp, synthetic construct
  source 1..73: mol_type = other DNA
tggatttggatgtgggatccggttcttggtgtcatccagccttagcaaaccagaggtaagaattttctgttaa

SEQ ID NO: 55 - DNA, 71 bp, synthetic construct
  source 1..71: mol_type = other DNA
tgggatttggatgtgggatccggttggtgtcatccagccttagcaaaccagaggtaagaattttctgttaa

SEQ ID NO: 56 - DNA, 72 bp, synthetic construct; identical to SEQ ID NO: 47
  source 1..72: mol_type = other DNA

SEQ ID NO: 57 - DNA, 66 bp, synthetic construct
  source 1..66: mol_type = other DNA
tgggatttggatgtgggatccggtttcatccgccttagcaaaccagaggtaagaattttctgttaa

SEQ ID NO: 58 - DNA, 69 bp, synthetic construct; identical to SEQ ID NO: 49
  source 1..69: mol_type = other DNA

SEQ ID NO: 59 - DNA, 76 bp, synthetic construct
  source 1..76: mol_type = other DNA
tgggatttggatgtgggatccggttnccttggtgtcatccagccttagcaaaccagaggtaagaattttctgttta

SEQ ID NO: 60 - DNA, 57 bp, synthetic construct
  source 1..57: mol_type = other DNA
tgggatttggatgtggcatccagccttagcaaaccagaggtaagaattttctgttaa

SEQ ID NO: 61 - DNA, 57 bp, synthetic construct
  source 1..57: mol_type = other DNA
tgggatttggatgtgggatccagccttagcaaaccagaggtaagaattttctgttaa

SEQ ID NO: 62 - DNA, 74 bp, synthetic construct
  source 1..74: mol_type = other DNA
tggatttggatgtgggatccggttnnnnnnccttggtgtcatccagccttagcaaaccagaggtaagaattttc

SEQ ID NO: 63 - DNA, 75 bp, synthetic construct
  source 1..75: mol_type = other DNA
tgggatttggatgtgggatccggttnnnccttggtgtcatccagccttagcaaaccagaggtaagaattttctgt

SEQ ID NO: 64 - DNA, 68 bp, synthetic construct
  source 1..68: mol_type = other DNA
tgggatttggatgtgggatcctggtgtcatccagccttagcaaaccagaggtaagaattttctgttaa

SEQ ID NO: 65 - DNA, 66 bp, synthetic construct
  source 1..66: mol_type = other DNA
tgggatttggatgtgggatccgggtcatccagccttagcaaaccagaggtaagaattttctgttaa

SEQ ID NO: 66 - DNA, 75 bp, synthetic construct
  source 1..75: mol_type = other DNA
tgggatttggatgtgggatccggttccttggtgtcatccagccttagcaaaccagaggtaagaattttctgttaa

SEQ ID NO: 67 - DNA, 42 bp, synthetic construct
  source 1..42: mol_type = other DNA
tgggatttggatgtgggatccggtttggtgtcatccagcctt

SEQ ID NO: 68 - DNA, 43 bp, synthetic construct
  source 1..43: mol_type = other DNA
tgggatttggatgtgggatccggttttggtgtcatccagcctt

SEQ ID NO: 69 - DNA, 39 bp, synthetic construct
  source 1..39: mol_type = other DNA
tgggatttggatgtgggatccttggtgtcatccagcctt

SEQ ID NO: 70 - DNA, 45 bp, synthetic construct
  source 1..45: mol_type = other DNA
tgggatttggatgtgggatcaggttgcttggtgtcatccagcctt

SEQ ID NO: 71 - DNA, 39 bp, synthetic construct
  source 1..39: mol_type = other DNA
tgggatttggatgtgggatccggggtgtcatccagcctt

SEQ ID NO: 72 - DNA, 40 bp, synthetic construct
  source 1..40: mol_type = other DNA
tgggatttggatgtgggatcaggttgcttggtgtcatcca

SEQ ID NO: 73 - DNA, 38 bp, synthetic construct
  source 1..38: mol_type = other DNA
tgggatttggatgtgggatccggttttggtgtcatcca

SEQ ID NO: 74 - DNA, 39 bp, synthetic construct
  source 1..39: mol_type = other DNA
tgggatttggatgtgggatccggttcttggtgtcatcca

SEQ ID NO: 75 - DNA, 35 bp, synthetic construct
  source 1..35: mol_type = other DNA
tggatttggatgtgggatccggttggtgtcatcca

SEQ ID NO: 76 - DNA, 37 bp, synthetic construct
  source 1..37: mol_type = other DNA
tgggatttggatgtgggatccggtttggtgtcatcca

SEQ ID NO: 77 - DNA, 32 bp, synthetic construct
  source 1..32: mol_type = other DNA
tgggatttggatgtgggatccggtttcatcca

SEQ ID NO: 78 - DNA, 34 bp, synthetic construct
  source 1..34: mol_type = other DNA
tgggatttggatgtgggatccttggtgtcatcca

SEQ ID NO: 79 - DNA, 40 bp, synthetic construct
  source 1..40: mol_type = other DNA
tgggatttggatgtgggatccggttnccttggtgtcatcc

SEQ ID NO: 80 - DNA, 22 bp, synthetic construct
  source 1..22: mol_type = other DNA
tgggatttggatgtggcatcca

SEQ ID NO: 81 - DNA, 22 bp, synthetic construct
  source 1..22: mol_type = other DNA
tgggatttggatgtgggatcca

SEQ ID NO: 82 - DNA, 40 bp, synthetic construct
  source 1..40: mol_type = other DNA
tgggatttggatgtgggatccggttnnnnnnccttggtgt

SEQ ID NO: 83 - DNA, 40 bp, synthetic construct
  source 1..40: mol_type = other DNA
tgggatttggatgtgggatccggttnnnccttggtgtcat

SEQ ID NO: 84 - DNA, 33 bp, synthetic construct
  source 1..33: mol_type = other DNA
tgggatttggatgtgggatcctggtgtcatcca

SEQ ID NO: 85 - DNA, 31 bp, synthetic construct
  source 1..31: mol_type = other DNA
tgggatttggatgtgggatccgggtcatcca

SEQ ID NO: 86 - DNA, 40 bp, synthetic construct
  source 1..40: mol_type = other DNA
tgggatttggatgtgggatccggttccttggtgtcatcca

SEQ ID NO: 87 - DNA, 33 bp, synthetic construct; identical to SEQ ID NO: 43
  source 1..33: mol_type = other DNA

SEQ ID NO: 88 - DNA, 33 bp, synthetic construct; identical to SEQ ID NO: 44
  source 1..33: mol_type = other DNA

SEQ ID NO: 89 - DNA, 33 bp, synthetic construct; identical to SEQ ID NO: 45
  source 1..33: mol_type = other DNA

SEQ ID NO: 90 - DNA, 33 bp, synthetic construct
  source 1..33: mol_type = other DNA
gctggatgacaccaagcaaccggatcccacatc

SEQ ID NO: 91 - DNA, 33 bp, synthetic construct
  source 1..33: mol_type = other DNA
ctcttccagtgggaccgagaccgtgaaagcatg

SEQ ID NO: 92 - DNA, 33 bp, synthetic construct
  source 1..33: mol_type = other DNA
catgctttcacggtctcggtcccactggaagag

SEQ ID NO: 93 - DNA, 33 bp, synthetic construct
  source 1..33: mol_type = other DNA
ctcttccagtgggaccaagaccgtgaaagcatg

SEQ ID NO: 94 - DNA, 33 bp, synthetic construct
  source 1..33: mol_type = other DNA
catgctttcacggtcttggtcccactggaagag